(19) United States
(12) Patent Application Publication
(10) Pub. No.: US 2018/0021297 A1
(43) Pub. Date: Jan. 25, 2018

(54) Use Of Croton- Or Calophyllum-Derived Proanthocyanidin Polymers Or Botanical Extracts In Combination With Rifaximin For The Treatment Of Diarrhea In Non-Human Animals

(71) Applicant: JAGUAR HEALTH, INC., San Francisco, CA (US)

(72) Inventor: Michael Hauser, San Francisco, CA (US)

(21) Appl. No.: 15/551,670

(22) PCT Filed: Feb. 24, 2016
(86) PCT No.: PCT/US16/19337

§ 371 Date: Aug. 17, 2017

Related U.S. Application Data
(60) Provisional application No. 62/121,296, filed on Feb. 26, 2015.

(51) Int. Cl. A61K 31/353; A61K 31/437; A23K 20/121; A61K 9/00; A61K 9/06; A23K 20/195; A61K 36/47; A61K 9/50

(52) U.S. Cl. CPC A61K 31/353; A61K 36/47; A61K 31/437; A61K 9/5026; A61K 9/0053; A61K 9/06; A23K 20/195; A23K 20/121

(57) ABSTRACT

Provided are combination therapy methods and uses for treating diarrhea, particularly malabsorption diarrhea, in neonatal, young and adult non-human animals, particularly for treating diarrhea resulting from microbial infection of the animals, with a therapeutically effective amount of a proanthocyanidin polymer from Croton lechleri, in either enteric or non-enteric form, in combination with a therapeutically effective amount of the antimicrobial agent rifaximin.

### FIELD OF THE INVENTION

The invention relates to the treatment of diarrhea in non-human animals involving the use of enteric or non-enteric formulations of a proanthocyanidin polymeric composition isolated from the plant Croton spp. or Calophyllum spp., or with a latex, extract, or food supplement derived therefrom, in combination with an antimicrobial agent, particularly, rifaximin. More specifically, the combination is effective in treating secretory or watery diarrhea that affects animal from various causes, including, infection and environmental factors. The treatments are effective in reducing the severity and duration of diarrhea in non-human animals, as well as increasing their survivability.

### BACKGROUND OF THE INVENTION

A wide array of infectious and pathogenic agents including bacteria, viruses, and parasites cause diarrhea in non-human animals, particularly, domesticated livestock animals associated with farming, food, and labor. Many of these enteropathogens cause one or more adverse effects in the animals, such as severe intestinal lesions, dehydration, alterations in enzyme activity, and/or alterations in nutrient transport mechanisms. The clinical presentation of diarrhea caused by these agents may vary; some diarrheas are self-limiting, while others are associated with high morbidity or high mortality (R. E. Holland, 1990, Clin. Microbiol. Rev., 3(4):345-375). In addition to infectious and pathological causes, environmental and other external factors, such as stress, overexertion, changes in diet and routine, may cause diarrhea of various types in non-human animals.

Secretory diarrheas, also called watery diarrheas, are a major source of illness and mortality in both young and adult non-human animals and are characterized by the loss of both fluid and electrolytes through the intestinal tract, leading to serious and often life-threatening dehydration. Secretory diarrhea is caused by a variety of bacterial, viral and protozoal pathogens and also results from other non-infectious etiologies such as ulcerative colitis, inflammatory bowel disease, environmental and stress conditions, and cancers and neoplasias of the gastrointestinal tract. All types of diarrheal disease may have a secretory component.

Infectious diarrhea of non-human animals, particularly very young animals, is an extremely common and economically devastating condition confronted by the animal agriculture and animal husbandry industries. When encountered in a herd, acute infectious diarrhea is often difficult to manage, contain and cure, because of the large numbers of potential enteropathogens involved, the differences in natural immunity among animals within the herd, environmental conditions and stresses, nutritional factors, the dynamics of the animal population, management conditions, and a difficulty in determining an etiological diagnosis. As a consequence, such a diagnosis is frequently not established for a large percentage of cases of neonatal animal diarrheas. In addition, neonatal and young animals, such as calves, may be predisposed to diarrheas as a result of difficult births, exposure, poor maternal nutrition and/or health, poor mothering capabilities on the part of the dam, or a combination of these factors. When some or all of these conditions occur, the resistance of the calf to infectious diseases, for example, is lowered, and exposure to and invasion by infectious agents play pivotal roles in producing diarrhea.

Some of the most common types of infectious agents resulting in neonatal animal diarrhea, particularly in calves, include E. coli, e.g., E. coli K-99; rotavirus and coronavirus, cryptosporidia, Salmonella spp., Campylobacter jejuni. In some cases, such as infection by Salmonella, which is a human pathogen, human handlers of the animals and those who treat the animals, may also be at risk of infection and disease.

Diarrhea in non-human animals can also be due to noninfectious causes, such as environmental changes, energy deficiencies and vitamin shortages that may cause weakness and susceptibility to infection, particularly in very young animals prior to weaning. Environmental and sanitation conditions associated with the birth of newborn animals can also be associated with outbreaks of disease and resulting diarrhea. For example, an unclean environment, e.g., an accumulation of urine and manure in an area where animals are born and nursed, can lead to disease syndromes that are characterized by diarrhea. In addition, problems related to giving birth by adult females, such as difficult calving and insufficient colostrum, can lead to weak newborns and a lack of passive immunity provided by the colostrum. Thus, adverse conditions affecting both the mothers and their newly and recently born offspring can lead to outbreaks of diarrhea requiring treatment of the neonatal and young animals. Noninfectious diarrhea, while oftentimes not severe enough to cause death, can weaken both young and adult animals and make them more susceptible to infectious diarrhea, which can debilitate adult animals and contribute to young animal's ability to survive.

The available and commonly used treatments for diarrhea in young and adult non-human animals typically involve vital fluid replacement and electrolyte replenishment to counter or stop fluid and electrolyte loss. Other types of treatments include the administration of gut-lining protectants, e.g., bismuth, oral antibiotics, and agents that affect gut motility. Depending on the cause(s), timing, severity and course of diarrhea and/or its associated disease or condition, the various known treatments may or may not be effective, and the animals may or may not respond adequately.

Rifaximin is an antimicrobial agent (antibiotic) that belongs to the family of naphthalene-ringed ansamycins, e.g., Rifampicin, Rifamycin. Rifaximin possesses a broad antibacterial spectrum against gram-positive bacteria (e.g., Staphylococci and Streptococci; Corynebacterium sp.) and against gram-negative bacteria (E. coli, Pasteurella sp., Pseudomonas sp., Proteus sp.). In veterinary medicine, and particularly in cattle, rifaximin is typically intended for the treatment and prevention of mastitis during the dry period by intramammary route of administration and for the treatment of postpartem metritis by intrauterine route of administration. (Committee for Veterinary Medicinal Products, The European Agency for the Evaluation of Medicinal Products, Summary Report (3), May, 1998). Because rifaximin is not systemically absorbed, its role is limited outside of the enteric area. Consequently, the development of microbial resistance is much less compared with systemically absorbed antibiotics, in addition to fewer systemic adverse effects and drug interactions. In addition, the virtual nonabsorption of rifaximin results in its high bioavailability in the gastrointestinal (GI) tract, which represents a primary therapeutic target, especially for treating GI infections. Clinical uses of rifaximin in humans include the treatment of inflammatory bowel disease, small intestine bacterial overgrowth, colonic diverticular disease and hepatic encephalopathy. Rifaximin is approved in 33 countries for GI disorders in humans. It is used in the treatment of traveler's diarrhea caused by E. coli and hepatic encephalopathy, for which it received orphan drug status from the U.S. Food and Drug Administration in 1998.

Because the economic and humane impacts of diarrhea and its related conditions on the afflicted animals, their handlers and caregivers are so great, there is a compelling need for alternative, safe, and medically effective, as well as cost effective, treatments and remedies for diarrhea and its associated symptoms in non-human young and adult animals. The present invention addresses such a need.

### SUMMARY OF THE INVENTION

The present invention provides a combination therapy method for treating diarrhea, such as secretory diarrhea, of a non-human young or adult animal, in which the method involves administering to an animal in need thereof a pharmaceutically acceptable composition comprising an aqueous soluble proanthocyanidin from Croton lechleri in combination with an antimicrobial agent or antibiotic, wherein the composition is formulated in an effective amount to treat the diarrhea in the animal. In a particular aspect, the method involves orally administering to an animal in need thereof a pharmaceutically acceptable composition comprising an aqueous soluble proanthocyanidin from Croton lechleri in combination with the antimicrobial agent rifaximin, wherein the composition is formulated in an effective amount to treat the diarrhea in the animal. Another embodiment of the invention provides methods of treating diarrhea in a non-human animal, including a neonate, juvenile, or adult animal in need thereof by administration, preferably orally, of an effective amount of rifaximin, including an effective amount of rifaximin in the absence of an aqueous soluble proanthocyanidin polymer composition from Croton lechleri or other Croton or Calophyllum species. In an aspect, the methods effectively treat the symptoms associated with diarrhea of various causes in afflicted animals; the symptoms may include dehydration, weight loss and electrolyte loss. In an embodiment, the diarrhea is episodic.

In embodiments of the above method, the pharmaceutically acceptable C. lechleri proanthocyanidin composition is an enteric formulation. In other embodiment, the pharmaceutically acceptable C. lechleri proanthocyanidin composition is an non-enteric formulation. In embodiments of the method, the C. lechleri proanthocyanidin polymer composition is SB 300, SP 303, or crofelemer. In an embodiment, the non-human animal is a non-adult, e.g., neonatal or young animal, or an adult animal. The method of the invention is not particularly limited as to the species of the animal and can include, exotic, zoo, farm, or domestic animals, e.g., without limitation, bovine, equine, ovine, porcine, or swine animals, goats, bison, buffalo, or camels.

In an embodiment, the invention provides a method for treating diarrhea in a non-human young or adult animal comprising administering to the animal a combination therapy comprising a C. lechleri-derived proanthocyanidin polymer, proanthocyanidin polymer composition, or botanical extract and an antimicrobial agent. In a preferred embodiment, the antimicrobial agent is rifaximin.

In an embodiment, the invention provides a pharmaceutical composition or formulation (also called a medicament herein) comprising a C. lechleri-derived proanthocyanidin polymer, proanthocyanidin polymer composition, or botanical extract for use in combination with rifaximin for treating diarrhea in a non-human young or adult animal.

In an embodiment, the invention provides a pharmaceutical composition or formulation comprising rifaximin for use in combination with a C. lechleri-derived proanthocyanidin polymer, proanthocyanidin polymer composition, or botanical extract for treating diarrhea in a non-human young or adult animal.

In an embodiment, the invention provides a pharmaceutical composition or formulation comprising a C. lechleri-derived proanthocyanidin polymer, proanthocyanidin polymer composition, or botanical extract and rifaximin for treating diarrhea in a non-human young or adult animal.

In other embodiments, the invention provides the use of a C. lechleri-derived proanthocyanidin polymer, proanthocyanidin polymer composition, or botanical extract in the manufacture of pharmaceutical composition or formulation for treating diarrhea in a young or adult non-human animal when administered in combination with rifaximin, as well as the use of rifaximin in the manufacture of a pharmaceutical composition or formulation for treating diarrhea in a young or adult non-human animal when administered in combination with a C. lechleri-derived proanthocyanidin polymer, proanthocyanidin polymer composition, or botanical extract. In further embodiments, the invention provides the use of a C. lechleri-derived proanthocyanidin polymer, proanthocyanidin polymer composition, or botanical extract and rifaximin in the manufacture of pharmaceutical composition or formulation for treating diarrhea in a young or adult non-human animal.

In an embodiment, the invention provides a kit which comprises a first container, a second container and a package insert or label information, wherein the first container comprises at least one dose of a pharmaceutical composition, formulation, or medicament comprising a C. lechleri-derived proanthocyanidin polymer, proanthocyanidin polymer composition, or botanical extract (e.g., SB 300 or SP 303, enteric or non-enteric coated); the second container comprises at least one dose of a pharmaceutical composition, formulation, or medicament comprising rifaximin; and the package insert or label information contains instructions for use in treating diarrhea in a young or adult non-human animal using the therapeutic agents and contents of the kit.

In other embodiments of the above methods, the C. lechleri proanthocyanidin polymer or a composition comprising the C. lechleri proanthocyanidin polymer is administered to the animal in an amount of at least 50 mg to 250 mg in combination with rifaximin in a therapeutically effective dose, for example, 10 mg to 1000 mg, 100 mg to 1000 mg, preferably from 20 mg to 750 mg, or 200 mg or 550 mg, or 200 mg to 600 mg. Rifaximin alone is administered in a therapeutically effective dose, for example, 10 mg to 1000 mg, 100 mg to 1000 mg, preferably from 20 mg to 750 mg, or 200 mg or 550 mg, or 200 mg to 600 mg. The dosage may depend upon the weight of the animal. The dosage of rifaximin may be anywhere from 0.1 mg/kg to 100 mg/kg, including 1 mg/kg, 2 mg/kg, 3 mg/kg, 4 mg/kg, 5 mg/kg, 10 mg/kg, or 20 mg/kg. In an embodiment, the C. lechleri proanthocyanidin polymer, or a composition comprising the C. lechleri proanthocyanidin polymer, is administered to the animal in an amount of at least 250 mg twice daily in combination with rifaximin. In still other embodiments of the method, the C. lechleri proanthocyanidin polymer composition is administered as powder reconstituted with a liquid selected from oral electrolytes, milk, milk replacer, physiological saline, or water in combination with rifaximin. In other embodiments of the method, the C. lechleri proanthocyanidin polymer composition is administered as a bolus, such as a pill, tablet or capsule in combination with rifaximin. In an embodiment, the C. lechleri proanthocyanidin polymer composition is orally administered in combination with rifaximin, which is also orally administered. In addition, the C. lechleri proanthocyanidin polymer composition may be administered in animal feed or drink in combination with rifaximin. In other embodiments of the method, the non-human neonatal, young, or adult animal is additionally suffering from an infection or disease associated with one or more of bacteria, parasites, viruses, or protozoa.

In an embodiment, the proanthocyanidin polymer is a formulation, composition, or extract from Croton lechleri, in combination with rifaximin. In an embodiment the proanthocyanidin polymer is a formulation, composition, or botanical extract from Croton lechleri administered in combination with rifaximin. In an embodiment, the formulation, composition, or botanical extract from Croton lechleri is in the form of a paste, gel, or gel paste. In an embodiment, the formulation, composition, or botanical extract from Croton lechleri is in the form of a gel, paste, or gel-paste in combination with rifaximin. For the combination therapy of the invention, the C. lechleri proanthocyanidin polymer composition (including in combination with rifaximin) is in the form of a gel, paste, or gel paste, which is typically administered to the animal by topical application to the roof of the animal's mouth. In some embodiments of the method, the gel, paste, or gel paste comprises polymeric microparticles or nanoparticles containing the C. lechleri proanthocyanidin polymer composition in combination with rifaximin. In an embodiment, the polymeric microparticles or nanoparticles are pH-sensitive. In some cases, the gel, paste, or gel paste is contained in a delivery device, which can be a syringe, such as a needle-less syringe.

In other embodiments, the proanthocyanidin polymer from Croton lechleri is a more highly purified composition containing proanthocyanidin polymer or oligomer, such as crofelemer or SB 300 compositions described herein. In a particular embodiment, the paste formulation comprises beads (nano or microparticles) comprising enterically coated SB 300 or SP 303 and is orally administered to animals in need. In an embodiment, the paste formulation comprises beads (nano or microparticles) comprising enterically coated SB 300. In a particular embodiment, the paste comprising SB 300 enteric beads is orally administered to an animal twice daily for three days, in combination with rifaximin. In some embodiments, the paste is orally administered for three consecutive days. In an embodiment, the paste comprising SB 300 enteric beads is orally administered to an animal in need at a dose of 2 mg/kg twice daily for three days, in combination with rifaximin.

In an embodiment, a composition or formulation of the invention contains an effective amount of a proanthocyanidin polymer formulation, composition, or botanical extract from Croton lechleri and an effective amount of rifaximin. In an embodiment, a proanthocyanidin polymer formulation, composition, or botanical extract from Croton lechleri is administered to an animal in need thereof in combination with the administration of rifaximin to the animal. In an embodiment, both the proanthocyanidin polymer formulation, composition, or botanical extract from Croton lechleri and rifaximin comprise the formulation or composition that is administered to the animal in need of treatment. In another embodiment, the proanthocyanidin polymer formulation, composition, or botanical extract from Croton lechleri may be administered to the animal in need separately from the administration of rifaximin.

In some aspects, rifaximin may be administered to a non-human animal in need to treat or prevent diarrhea, without the co-administration of a proanthocyanidin polymer formulation, composition, or botanical extract from Croton lechleri. In a particular embodiment, rifaximin is orally administered in an effective amount for treating or preventing diarrhea of various causes, including microbial infection, in a non-human young or adult animal. In embodiments, the therapeutic effects of rifaximin in treating diarrhea in a non-human animal in need, or in protecting an animal from diarrhea, are provided by rifaximin with or without the administration of a C. lechleri-derived proanthocyanidin polymer, composition, or botanical extract. In an embodiment, rifaximin is administered to the animal in a solid dosage form. In an embodiment, rifaximin is orally administered.

In embodiments in which a combination of the proanthocyanidin polymer formulation, composition, or botanical extract from Croton lechleri and rifaximin is administered to the animal in need thereof as separate formulations or compositions, the proanthocyanidin polymer formulation, composition, or botanical extract from Croton lechleri may be administered at the same time as the administration of rifaximin, or at a predetermined time before or after the administration of rifaximin to the animal, for example, prior to or after the administration of rifaximin. In an embodiment, a C. lechleri proanthocyanidin polymer formulation, composition, or botanical extract is administered immediately before or after the administration of rifaximin to the animal, and vice versa. In some embodiments, an interval of time, e.g., several minutes up to an hour, may separate the administration of the proanthocyanidin polymer formulation, composition, or botanical extract from Croton lechleri and the administration of rifaximin, and vice versa, to the animal. In an embodiment, rifaximin is administered to the animal in a therapeutic dosage form, such as a solid dosage form or an oral solid dosage form, in combination with a C. lechleri proanthocyanidin polymer, composition, or botanical extract, e.g., SB 300 or SP 303, in either the same therapeutic dosage form as rifaximin, or in another dosage form that is administered in combination with the rifaximin dosage form.

In various aspects, the combination therapy methods of the invention are advantageous and effective for treating and controlling diarrhea in non-human young and adult animals that are affected and for improving and maintaining the animals' gut health and overall strength and fitness. This is especially the case because the C. lechleri proanthocyanidin polymer, composition, or botanical extract component, as well as the rifaximin component, of the combination therapy administered, particularly, orally administered, in the diarrhea treatments of the invention remain substantially localized in the enteric environment of the gut where their antidiarrheal effects are active and manifest, especially against diarrhea-causing gut pathogens (bacteria). By way of example, rifaximin, which provides broad spectrum antibacterial activity and is not systemically absorbed following oral administration, allows localized enteric targeting of diarrhea-causing pathogens in the GI tract of the animal with a minimal-to-no risk of systemic dissemination, toxicity, or side effects.

In an aspect, the invention provides a method of treating diarrhea in a non-human animal in which a combination therapy comprising an aqueous soluble proanthocyanidin polymer from Croton lechleri, or a C. lechleri botanical extract and the antimicrobial agent rifaximin is orally administered to the non-human animal in need thereof in amounts effective to treat the diarrhea in the non-human animal. In an embodiment, the combination of rifaximin and a C. lechleri proanthocyanidin polymer, composition, or botanical extract thereof provides localized and targeted activity against diarrhea-causing pathogens in the enteric environment of the GI tract of the animal undergoing treatment and is not systemically absorbed. In an embodiment, animal materials and consumable products derived or produced from a non-human animal treated with the combination of rifaximin and a C. lechleri proanthocyanidin polymer, composition, or botanical extract thereof may contain insignificant or less than expected residual amounts of the rifaximin and/or the C. lechleri proanthocyanidin polymer, composition, or botanical extract components, or breakdown products thereof. In an embodiment, the virtual lack of systemic absorption of the components of the treatment methods of the invention advantageously may minimize or eliminate resistance to the rifaximin, the C. lechleri proanthocyanidin polymer, composition, or botanical extract components of the combination therapy, or breakdown products thereof, in the treated non-human animals, as well as in materials and consumable products derived therefrom.

### DETAILED DESCRIPTION OF THE INVENTION

The methods of the present invention provide a new combination therapy for treating diarrhea in non-human animals, in particular, diarrhea caused microbial infection of afflicted animals. The methods of the invention provide treatment of diarrhea resulting from microbial infection of non-human animals with an effective amount of a combination therapy involving a polymeric proanthocyanidin composition from a Croton species or Calophyllum species (e.g., SB 300 or SP 303), or with a latex, extract or food supplement botanical extract derived therefrom, in conjunction with the antimicrobial agent rifaximin. The effective treatment of diarrhea in afflicted non-human animals with a proanthocyanidin polymer composition from Croton lechleri, or with a latex, extract or food supplement botanical extract derived therefrom, in combination with rifaximin, offers a high level of therapeutic and antimicrobial activity that optimally may remain localized and bioavailable within the enteric environment of the GI tract of the animal in order to treat the diarrhea with little to no systemic dissemination. The invention further provides methods of treating diarrhea in non-human animals by administration of an effective amount of rifaximin in the absence of an aqueous soluble proanthocyanidin polymer composition derived from a Croton species or Calophyllum species. The methods further provide the benefit of treating diarrhea with little to no systemic dissemination of the therapeutic agent or any breakdown products thereof.

The mechanism of action of C. lechleri- or Calophyllum-derived polymeric proanthocyanidin compositions, or purified extracts such as crofelemer, is through the inhibition of both the cystic fibrosis transmembrane conductance regulator protein (CFTR) chloride ion channel and the calcium-activated chloride ion channels (CaCC). The polymeric proanthocyanidin composition acts by blocking chloride ion channel secretion and the accompanying high volume water loss occurring in diarrhea, thus normalizing the flow of chloride ions and water in the gastrointestinal (GI) tract. Because rifaximin is virtually nonabsorbed, its bioavailability and antimicrobial activity within the GI tract is quite high, thus providing intraluminal and fecal drug concentrations that are effective against a wide range of pathogenic organisms associated with GI infections in animals.

The invention provides methods directed to treating the debilitating problem of diarrhea and microorganism-induced diarrhea in unweaned, young and adult non-human animals. The methods are effective in reducing and/or alleviating diarrhea in such non-human animals in need thereof. In particular, the methods are directed to the treatment of diarrhea, particularly secretory/watery diarrhea, of various causes, including microbial infection, alone or in combination with other infectious agents or environmental conditions, in adult animals and in neonatal and young (juvenile, non-adult) animals, particularly where scourges of diarrhea in such animals can have a profound economic impact for the animal agriculture, food and health industries.

The invention further provides formulations and compositions containing a proanthocyanidin polymer composition from Croton lechleri, or with a latex, extract or food supplement botanical extract derived therefrom, in combination with rifaximin suitable for treating diarrhea in neonatal, young and adult animals. Unless otherwise noted herein, use of the term “animal” herein denotes non-human, warm-blooded mammals of a number of different species. In addition, the terms “young”, non-adult”, “immature” and “juvenile” are used synonymously herein. Without wishing to be limiting, “young” animals are generally under one year of age. “Neonatal” animals are generally two weeks of age or less.

The methods of the invention provide a solution to a significant need for the animal industry, e.g., the beef and dairy industries worldwide, particularly for countries in which neonatal calf diarrhea presents one of the largest health challenges, as well as economic losses.

The methods and treatments of the invention are particularly suitable for treating animals of a young age, as well as adult animals, that have a bacterial infection. In an embodiment, the animals are neonatal (or newborn), unweaned, non-adult animals that are born, bred, raised and/or maintained in a domesticated and/or agricultural setting, e.g., as livestock and farm animals, for commodities such as food, labor, sport, show, or other commercial or non-commercial agricultural husbandry capacity. In an embodiment, the animals are adult animals in the aforementioned settings. Nonlimiting examples of animals affected by diarrhea and treatable by the methods and formulations of the invention include, without limitation, neonatal and young cattle (calves), pigs (piglets), sheep (lambs), goats (kids), horses (foals) and camels (calves), and adult animals, including, cattle, steer, bison, buffalo, horses, camels, goats, sheep and rams, as further described herein. In an embodiment, the neonatal, young, or adult animals are rabbits, domestic, companion animals, such as, without limitation, dogs and cats of any species, small rodents, guinea pigs, hamsters, gerbils, rats, mice and the like. As used herein, the terms “neonatal” and “newborn” are synonymous.

In particular embodiments, non-human animals may be administered the proanthocyanidin polymer compositions in combination with rifaximin according to the invention to prevent diarrhea in an animal or population of animals exposed to, at risk of being exposed to, or suspected of being exposed to microbial, for example, in connection with a microbial or bacterial infection outbreak in a herd, population, or other group of animals held in close proximity.

The present invention particularly relates to treating diarrhea in adult, or in neonatal, unweaned and young, animals with physiologically and pharmaceutically acceptable formulations and compositions comprising a therapeutically effective amount of an antidiarrheal agent comprising a proanthocyanidin polymer obtained from a Croton spp., preferably Croton lechleri (C. lechleri) in combination with rifaximin. In embodiments, the proanthocyanidin polymer composition can also be obtained from a Calophyllum spp., in particular Calophyllum inophylum. In an specific embodiment, the pharmaceutically acceptable composition comprises a proanthocyanidin polymer from Croton lechleri. In a particular embodiment, the proanthocyanidin polymer is enterically protected beads, including enteric beads including SB 300 or SP 303. As indicated supra, rifaximin may be a component of such physiologically and pharmaceutically acceptable, C. lechleri-derived, proanthocyanidin polymer formulations and compositions, or rifaximin may be administered separately, but in combination with, the administration of the C. lechleri-derived, proanthocyanidin polymer. In various embodiments, rifaximin is orally administered to an animal in need and/or C. lechleri-derived proanthocyanidin polymer formulations, compositions, or botanical extracts are orally administered.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve alleviating one or more symptoms of a disease or condition, although not necessarily all of the symptoms, of the disease or condition, or attenuating the symptoms or progression of the disease or condition. Curing or eliminating the disease or condition from the animal is an optimal outcome of the practice of the methods of the invention.

According to the invention, treatment of an animal in need thereof typically involves the use or administration of an effective amount or a therapeutically effective amount of a proanthocyanidin polymer or a proanthocyanidin polymer composition preferably from a Croton spp. particularly C. lechleri, either enteric or non-enteric, in combination with rifaximin. Effective amount refers to the quantity (amount) of the composition, and the like, that induces a desired response in the animal subject upon administration or delivery to the animal. Optimally, an effective amount produces a therapeutic effect in the absence of, or with little or virtually no, adverse effects or cytotoxicity in the animal. Alternatively, any adverse effects associated with an effective amount are optimally outweighed by the therapeutic benefit achieved. It is to be understood that rifaximin is also used and administered, e.g., orally administered, in an effective amount or in a therapeutically effective amount that induces a desired response in the non-human animal subject upon administration or delivery to the animal.

In an aspect, the administration of rifaximin and a C. lechleri proanthocyanidin polymer, composition, or botanical extract as active agents in the combination therapy and uses of the invention may provide an interactive or synergistic effect in treating diarrhea in a non-human young or adult animal in need. In an embodiment, diarrhea caused by bacterial pathogens of the gut is treated in accordance with the invention. Without wishing to be bound by theory, rifaximin in the combination treatment may increase or positively modulate the effectiveness of the C. lechleri proanthocyanidin polymer, composition, or botanical extract to provide an enhanced, augmented, improved, increased, or heightened treatment effect compared with the effect of either agent alone. As well, the C. lechleri proanthocyanidin polymer, composition, or botanical extract in the combination treatment may increase or positively modulate the effectiveness of rifaximin to achieve an enhanced, augmented, improved, increased, or heightened treatment effect compared with the effect of either agent alone.

In some aspects, rifaximin may be administered to a non-human animal in need to treat or prevent diarrhea, without the co-administration of a proanthocyanidin polymer formulation, composition, or botanical extract from Croton lechleri. In a particular embodiment, rifaximin is orally administered in an effective amount for treating or preventing diarrhea of various causes, including microbial infection in the gut, in a non-human young or adult animal. In embodiments, the therapeutic effects of rifaximin in treating diarrhea in a non-human animal in need, or in protecting an animal from diarrhea, are provided by rifaximin with or without the administration of a C. lechleri-derived proanthocyanidin polymer, composition, or botanical extract. In an embodiment, rifaximin is administered to the animal in a solid dosage form. In an embodiment, rifaximin is orally administered.

The treatment methods are directed to ameliorating, preventing, inhibiting, reversing, attenuating, alleviating, abrogating, controlling, minimizing, suppressing, reducing, decreasing, diminishing, stabilizing, eradicating, curing, or eliminating diarrhea and/or its associated symptoms, especially diarrhea caused by microbial infection that adversely affects the health, growth and survivability of neonatal and young, as well as adult, non-human animals. In an embodiment, the diarrhea is secretory/watery diarrhea. Such diarrhea can be a clinical sign of gastrointestinal (GI) disease in an animal; it can also reflect primary disorders outside of the digestive system, such as disorders affecting the large bowel or the small bowel. The methods described herein are also suitable for treating diarrhea resulting from microbial infection in animals that may have comorbid conditions or disorders that cause diarrhea via different mechanisms involved in their pathogenesis, for example, osmotic diarrhea, secretory diarrhea, episodic diarrhea, or inflammatory and infectious diarrhea. In an embodiment, the neonatal or young animal can suffer from diarrhea associated with inflammation of the lining of the colon, such as colitis, or acute colitis, which can be caused by infection or inflammation of the bowel.

Osmotic diarrhea is associated with absorption of water in the intestines, which depends upon adequate absorption of solutes. If excessive amounts of solutes are retained in the intestinal lumen, water will not be absorbed and diarrhea results. Osmotic diarrhea typically results from ingestion of a poorly absorbed substrate, for example, a carbohydrate or divalent ion or from malabsorption of any type, such as an inability to absorb certain carbohydrates. Secretory diarrhea occurs when the secretion of water into the lumen of the intestine exceed absorption. Under normal conditions, large volumes of water are secreted into the small intestinal lumen, but a large portion of this water is efficiently absorbed before reaching the large intestine.

Secretory diarrhea can result from exposure of an animal to toxins (enterotoxins) from certain types of bacteria, such as cholera toxin of Vibrio cholerae and heat-labile toxin of E. coli. Massive diarrhea is induced from such microorganisms as a consequence of their toxins strongly activating adenylyl cyclase, which causes a prolonged increase in the intracellular concentration of cyclic AMP within crypt enterocytes. This increase, in turn, results in prolonged opening of the chloride channels that contributes to secretion of water from the crypts, thereby allowing uncontrolled secretion of water. These bacterial toxins can also affect the enteric nervous system, resulting in an independent stimulus of water secretion.

Inflammatory and infectious diarrhea can be caused by the disruption of the epithelium of the intestine due to microbial or viral pathogens. Typically, the epithelium of the digestive tube is protected from insult by a number of mechanisms that constitute the gastrointestinal barrier. However, the gastrointestinal barrier can be breached and result in diarrhea. Destruction of the epithelium results not only in leaking of serum and blood into the lumen but also is often associated with significant destruction of adsorptive epithelium. When this occurs, the absorption of water becomes highly inefficient and diarrhea results. The pathogenic culprits frequently associated with infectious diarrhea include bacteria, such as E. coli, Campylobacter and Salmonella; viruses, such as rotaviruses, coronaviruses, parvoviruses and norovirus; and protozoa, such as coccidia species, Cryptosporium and Giardia. In addition, the response of the immune system to inflammatory conditions in the bowel contributes greatly to the development of diarrhea. Activated white blood cells are stimulated to produce and secrete inflammatory mediators and cytokines that stimulate secretion. An secretory component is thus imposed upon and exacerbates an inflammatory diarrhea. Moreover, reactive oxygen species produced by leukocytes can damage or destroy intestinal epithelial cells, which are replaced with immature cells that are generally lacking in the brush border enzymes and transporters necessary for the absorption of nutrients and water. Thus, components of an osmotic (malabsorption) diarrhea provide additional pathology and problems for an afflicted animal.

In a particular embodiment, the diarrhea to be treated is caused by infection or invasion of the animals by microbial (bacterial) pathogens. In other embodiments, the animal may suffer from diarrhea caused by environmental agents and/or diarrhea resulting from one or more infectious agents, including bacteria, e.g., Escherichia coli, Salmonella sp., Clostridium perfringes, C. difficile, Staphylococcus aureus, H. pylori, etc.; viruses, e.g., coronaviruses, rotaviruses, bovine virus diarrhea (BVD) virus, infectious bovine rhinotracheitis (IBR) virus, etc.; protozoa, e.g., Cryptosporidium, coccidia, etc.; as well as yeasts and molds. The methods and compositions of the invention are suitable for treating animals afflicted with such types of mixed infections.

The treatment of diarrhea in neonatal and young animals according to the combination therapy of the invention is of particular importance, because such immature animals are most susceptible to infection by bacteria, as well as numerous other types of pathogens, and resistance to infection develops with increasing age of the animal. In addition, younger animals experience more severe clinical illness as a result of infection and resulting diarrhea. In general, due to the anatomy of the gastrointestinal tract of adult animals such as horses, conditions affecting the large intestine and cecum typically cause diarrhea. However, young animals, e.g., foals, that are less than about two or three months of age do not have fully competent large intestines and ceca as do adult animals; therefore, young animals tend to be more prone to diarrhea caused by small intestinal conditions. In general terms, a foal is an equine, particularly a horse, that is one year old or younger in age.

In some cases, in addition to bacteria-induced diarrheal disease, noninfectious causes can augment or worsen the problems experienced by the animals, for example, without limitation, inadequate nutrition and/or insufficient attention of a neonate or young animal on the part of the mother, or in both young and adult animals, exposure to severe environment, or a combination of these events. In another embodiment, diarrhea results from a combination of the invasion of infectious microorganisms and noninfectious factors. Frequently, noninfectious causes of diarrhea in young or adult animals are considered to be factors that predispose or contribute to an animal's susceptibility to infectious agents and causes of diarrhea. Whether the cause of diarrhea in animals is infectious or noninfectious, the absorption of fluids from the intestine is altered and life-threatening electrolyte imbalances can occur. The affected animals lose fluids, rapidly dehydrate and suffer from electrolyte loss and acidosis. Although infectious agents may cause an initial damage to the animal's intestine, actual death from diarrhea (serious diarrhea) in animals usually is a consequence of dehydration, acidosis and loss of electrolytes, which may be difficult to replenish in adequate amount and time. Accordingly, the combination therapy methods and formulations of the invention are suitable for treating diarrhea and the symptoms of diarrhea, such as dehydration and electrolyte loss, in an effort to prevent more severe dehydration and animal death.

Proanthocyanidins and Tannins Obtained from Plant Extracts

Proanthocyanidins are types of condensed tannins, which are found in a large number of plants and are classified as hydrolyzable or condensed. Tannins and, in particular, proanthocyanidins are contained in many plants used in traditional medicine as treatment or prophylaxis for diarrhea (See, e.g., Yoshida et al., 1993, Phytochemistry, 32:1033; Yoshida et al., 1992, Chem. Pharm. Bull., 40:1997; Tamaka et al., 1992, Chem. Pharm. Bull., 40:2092).

Proanthocyanidins are comprised of at least two or more monomer units that may be of the same or different monomeric structure. The monomer units (generally termed “leucoanthocyanidins”) are generally monomeric flavonoids which include catechins, epicatechins, gallocatechins, galloepicatechins, flavanols, flavonols, flavan-3,4-diols, leucocyanidins and anthocyanidins. The polymer chains are thus based on different structural units, creating a wide variation of polymeric proanthocyanidins and a large number of possible isomers (Hemingway et al., 1982, J. C. S. Perkin, 1:1217). Larger polymers of the flavonoid 3-ol units are predominant in most plants and often have average molecular weights above 2,000 daltons (Da), containing 6 or more units (Newman et al., 1987, Mag. Res. Chem., 25:118).

Proanthocyanidin polymers and proanthocyanidin are found in a wide variety of plants, especially those having a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, which are endemic to South America, produce a red viscous latex sap called Sangre de Drago or “Dragon's Blood”. The red viscous latex is known for its medicinal properties. For example, U.S. Pat. No. 5,211,944 describes the isolation of an aqueous soluble proanthocyanidin polymer composition from Croton spp. (See also, Ubillas et al., 1994, Phytomedicine, 1:77). The isolation of an aqueous soluble proanthocyanidin polymer composition from Calophyllum inophylum in U.S. Pat. No. 5,211,944.

In an embodiment, a proanthocyanidin polymer from C. lechleri, or a composition thereof, is crofelemer. Crofelemer (CAS 148465-45-6) is an oligomeric proanthocyanidin of varying chain lengths derived from the Dragon's Blood of Croton lechleri, a tree of the family Euphorbiaceae, which is sustainably harvested under fair trade work practices in the Amazon. It has an average molecular weight of approximately 1900 Da to approximately 2700 Da. The monomers comprising crofelemer comprise catechin, epicatechin, gallocatechin, and epigallocatechin. The chain length of crofelemer ranges from about 3 to about 30 units with an average chain length of about 8 units. Crofelemer has the chemical formula: (C₁₅O_{6,7}H₁₂)ₙ and a molecular mass of 860-9100 g/mol. The antisecretory mechanism of action of crofelemer involves the targeting and inhibition of two, distinct intestinal chloride channels, namely, the cystic fibrosis transmembrane regulator conductance (CFTR) channel, which is a cAMP-stimulated Cl⁻ channel, and the calcium-activated chloride channel (CaCC), as reported, for example, by Tradtrantip, L. et al., 2010, “Crofelemer, an Antisecretory Antidiarrheal Proanthocyanidin Oligomer Extracted from Croton lechleri, Targets Two Distinct Intestinal Chloride Channels”, Mol. Pharmacol., 77(1):69-78). A general structure of crofelemer is shown below. In the structure, an H at the R position of the structure signifies procyanidin; an OH at the R position of the structure signifies prodelphinidin.

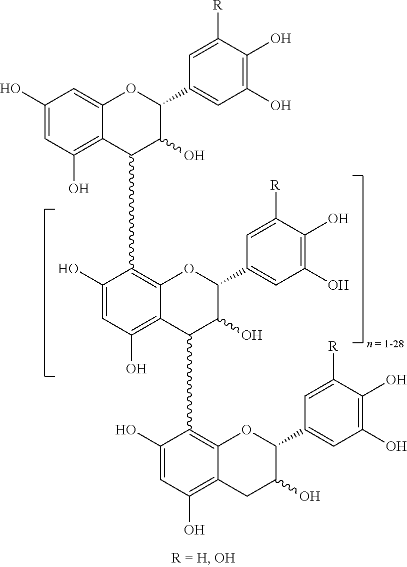

In accordance with an embodiment of the invention, crofelemer, or a pharmaceutically acceptable formulation or composition comprising crofelemer, is employed in the treatment methods as the proanthocyanidin polymer from Croton lechleri.

In an embodiment, SP 303, an oligomeric proanthocyanidin from Croton lechleri, (also known as crofelemer) is the proanthocyanidin polymer from Croton lechleri, or a pharmaceutically acceptable formulation or composition comprising SP 303, which is suitable for use in the treatment methods of the invention. SP-303 (R. Ubillas et al., 1994, Phytomedicine, 1:77-106) is largely composed of purified proanthocyanidin oligomers (−)-galloepicatechin and (+)-gallocatechin, (−)-epicatechin and (+)-catechin and is suitable for use in the enteric and non-enteric formulations and compositions for administration in the treatment methods described herein.

In another embodiment, SB 300, a proanthocyanidin polymer extract from Croton lechleri is the proanthocyanidin polymer from Croton lechleri, or a pharmaceutically acceptable formulation or composition comprising SB 300, which is suitable for use in the treatment methods of the invention. SB 300, as described, for example, by Fischer, H. et al., (2004, J. Ethnopharmacol., 93(2-3):351-357) provides a natural product extract that is particularly amenable for both enteric and non-enteric formulations and compositions, and is highly functional and cost-effective in the treatment methods described herein.

A pharmaceutically acceptable composition comprising a proanthocyanidin polymer from Croton lechleri and employed in the treatment methods of the invention can be obtained from C. lechleri, e.g., as described in WO 00/47062 to Shaman Pharmaceuticals, Inc., the contents of which are incorporated herein, and formulated as a food or dietary supplement or nutraceutical formulation.

In other embodiments, compositions useful in the methods of the invention comprise a raw latex obtained from a Croton species or a Calophyllum species, or an extract obtained from a Croton species or a Calophyllum species, which are not specifically polymeric proanthocyanidin polymer compositions. Exemplary extracts are described in Persinos et al., 1979, J. Pharma. Sci., 68:124 and Sethi, 1977, Canadian J. Pharm. Sci., 12:7.

In an embodiment, the proanthocyanidin polymer from Croton lechleri is formulated with an enteric coating or matrix in a variety of dosage formats known in the art (See, e.g., WO 00/47062 and U.S. Pat. Nos. 7,441,744 and 7,323,195, the contents of which are incorporated herein, and as briefly described below. In another embodiment, the proanthocyanidin polymer is formulation without an enteric coating or matrix. Both enteric and non-enteric forms of the proanthocyanidin polymer from Croton lechleri, for example, SB 300, are intended for use in the methods of the present invention.

### Preparation of Proanthocyanidin Polymer Compositions and Formulations

The proanthocyanidin polymer composition, effective for treating secretory diarrhea according to the invention, is comprised of monomeric units of leucoanthocyanidins. More particularly, the composition is comprised of proanthocyanidin polymers of 2 to 30 flavonoid units, preferably 2 to 15 flavonoid units, more preferably 2 to 11 flavonoid units and most preferably an average of 7 to 8 flavonoid units with a number average molecular weight of approximately 2500 Da. The proanthocyanidin polymer composition is preferably soluble in an aqueous solution. Preferred for use in the methods according to the invention is a proanthocyanidin polymer from C. lechleri; such a C. lechleri proanthocyanidin polymer may be in the form of a pharmaceutically acceptable composition.

Examples of proanthocyanidin polymeric compositions useful in the present invention are preferably isolated or purified from a Croton spp., namely, Croton lechleri, or Calophyllum spp. by any method known in the art. For example, the proanthocyanidin polymer composition may be isolated from a Croton spp. or Calophylum spp. by the method disclosed in U.S. Pat. No. 5,211,944 or in Ubillas et al. (1994, Phytomedicine, 1:77-106, called SP 303 therein), both of which are incorporated herein by reference. Other isolation methods are described in U.S. Pat. Nos. 7,556,831 and 8,067,041 (Example 2), the contents of which are incorporated herein. PCT Application No. PCT/US00/02687, published as WO 00/47062, the contents of which are incorporated herein, also discloses a method of manufacturing a proanthocyanidin polymeric composition isolated from Croton spp. or Calophyllum spp. and enteric formulations of proanthocyanidin polymer dietary supplements, as well as methods of their preparation. Another illustrative method for isolating proanthocyanidin polymer from C. lechleri (such as crofelemer) is found in U.S. Pat. Nos. 7,341,744 and 7,323,195, the contents of which are expressly incorporated herein. As described above, the SP 303 and SB 300 purified forms of oligomeric proanthocyanidin polymer from Croton lechleri are suitable for use in the treatment methods of the invention.

In an embodiment, the proanthocyanidin polymer composition may be generally isolated by the following process, such as provided in U.S. Pat. No. 7,341,744, the contents of which are incorporated herein. Latex collected from Croton lechleri plants is mixed with purified water (preferably one part latex to two parts purified water). Any insoluble material in the latex solution is allowed to settle, e.g., by leaving the mixture at 4° C. overnight (12 hours). The supernatant is pumped away from the residue and is extracted with a short chain alcohol, such as n-butanol. The extraction is preferably performed multiple times, such as three times. After each extraction, the alcohol phase is discarded and the aqueous phase is retained. The aqueous phase is concentrated, for example, using an ultrafiltration device with a 1 kD cut-off membrane. This membrane can be a low protein binding cellulose membrane, or, alternatively, a polypropylene, teflon or nylon membrane can be used. The membrane used should be compatible with acetone. The purpose of the ultrafiltration is to remove the water from the material.

The retentate from the ultrafiltration is then concentrated to dryness, for example using tray-dryers at approximately 37° C. (±2° C.). The dried material is subsequently dissolved in water and is then chromatographed on a cation exchange column (e.g., a CM-Sepharose column) and a size exclusion column (e.g., an LH-20 column). In the preferred two column system, material is run over a CM-Sepharose and then an LH-20 column in a series. Specifically, the dissolved material is loaded onto the cation exchange column and is then washed with purified water. The proanthocyanidin polymer material is eluted from the cation exchange column with an aqueous acetone solution (preferably 30% acetone), thereby loading the proanthocyanidin polymer material onto the sizing column. The sizing column is disconnected from the cation exchange column and the material is then eluted off of the sizing column with an aqueous acetone solution (preferably 45% acetone). The fractions are collected and monitored with a UV detector, e.g., at a wavelength of 460 nm. Fractions containing the proanthocyanidin polymer material are combined and concentrated, for example, by ultrafiltration using, e.g., a 1 kD cut-off membrane (as described above for the ultrafiltration step prior to the chromatography steps). The retentate may then be concentrated to dryness using a suitable drying method, such as, but not limited to, a rotary evaporator, at a temperature of approximately 37° C. (±2° C.). Other suitable drying methodologies include, but are not limited to, tray drying and spray drying. Example 10 of U.S. Pat. No. 7,341,744 provides additional, non-limiting, methodology for preparing a composition comprising proanthocyanidin polymer, which can be used according to the invention. A detailed protocol for isolating an enriched proanthocyanidin polymer extract suitable for use in the methods of the invention is described in WO 00/47062 as noted herein above.

### Rifaximin

Rifaximin (4-deoxy-4′-methylpyrido[1′,2′-1,2]imidazo-[5,4-c]-rifamycin SV] is a semi-synthetic antimicrobial agent that has been used in the treatment of diarrhea caused by noninvasive strains of Escherichia coli, such as traveler's diarrhea and hepatic encephalopathy in human patients. Because rifaximin is not systemically absorbed, it results in the development of less resistance compared with systemically absorbed antibiotics, in addition to fewer systemic adverse effects and drug interactions. Rifaximin, which possesses a broad antibacterial spectrum against gram-positive and gram-negative bacteria, is active against diarrhea-causing E. coli bacterial strains, inhibiting growth of the bacteria by preventing them from manufacturing proteins needed for their replication and survival. More specifically, rifaximin interferes with transcription by binding to the β-subunit of bacterial RNA polymerase. By suppressing growth of the bacteria, rifaximin reduces symptoms of diarrhea. Hepatic encephalopathy is a serious neurologic complication of advanced liver disease that affects the brain. It is believed to be caused by the absorption of ammonia and other chemicals produced by bacteria in the intestine. This results in the blockage of the translocation step that normally follows the formation of the first phosphodiester bond, which occurs in the transcription process. It is believed that rifaximin prevents and treats hepatic encephalopathy by reducing the intestinal bacteria that produce ammonia.

Rifaximin is a derivative of rifamycin, specifically, pyrido-imidazo rifamycin. Rifaximin and a method of making rifaximin is described in U.S. Pat. No. 4,557,866 and European Patent No. EP0161534, both of which are incorporated herein by reference. Purified and polymorph forms of rifaximin having low GI absorption and methods of preparation thereof are described in the art, for example, as found in U.S. Pat. Nos. 7,045,620; 7,612,199; 7,902,206; 7,906,542; 8,158,644; and 8,158,781, the contents of which are incorporated by reference herein. Solid dosage forms of rifaximin (available for medical use as Xifaxan) are film coated and contain 200 mg or 550 mg of rifaximin as active ingredient. Such tablets also comprise inactive ingredients, such as colloidal silicon dioxide, disodium edetate, glycerol palmitostearate, hypromellose, microcrystalline cellulose, propylene glycol, red iron oxide, sodium starch glycolate, talc and titanium dioxide. (Xifaxan product insert, NDA 21-361/S-006).

For the combination therapeutic methods of the invention as well as the methods in which rifaximin is administered in the absence of a proanthocyanidin polymer composition, rifaximin may be administered in solid dosage form, such as, without limitation, tablets of 200 mg and 550 mg, or as produced according to methods known in the art, such as those described in U.S. Pat. No. 4,557,866. In an embodiment, rifaximin is orally administered.

According to an aspect of the invention, the combination therapy methods and methods of administering rifaximin alone are advantageous and effective for treating, controlling and eliminating diarrhea, particularly, diarrhea caused by enteric, pathogenic bacteria, in non-human young and adult animals that are affected, and for improving and maintaining the animals' gut health and overall strength and fitness. Because rifaximin and the C. lechleri proanthocyanidin polymer, composition, or botanical extract components of the combination therapy administered in the diarrhea treatments of the invention remain substantially localized in the enteric environment of the gut of a treated animal, their antidiarrheal effects are also localized for targeting diarrhea-causing gut pathogens (bacteria). Neither component of the combination therapy of the invention is systemically absorbed to any appreciable extent, thus allowing targeting of diarrhea-causing pathogens essentially contained in and restricted to the enteric environment of the GI tract of the animal. The lack of systemic dissemination also provides a low or no amount of toxicity or adverse side effects in the animals undergoing treatment.

In an embodiment, the combination therapy of the invention comprising rifaximin and a C. lechleri proanthocyanidin polymer, composition, or botanical extract in amounts effective to control, reduce, and/or treat the diarrhea in non-human animals provides localized and effective antidiarrheal activity in the enteric environment/gut of a treated animal. Such localized, non-systemic activity of the rifaximin and C. lechleri proanthocyanidin polymer, composition, or botanical extract components, or components resulting from metabolism of the drugs, of the treatment methods ultimately results in less than expected and/or unappreciable amounts of component residue in animal-derived products, such as meat and/or milk products, following the treatment methods. In an embodiment, the therapy of the invention comprising administering rifaximin in the absence of C. lechleri proanthocyanidin polymer in an amount effective to control, reduce, and/or treat the diarrhea in non-human animals provides localized and effective antidiarrheal activity in the enteric environment/gut of a treated animal. Such localized, non-systemic activity of the rifaximin ultimately results in less than expected and/or unappreciable amounts of component residue, or component byproduct residue, in animal-derived products, such as meat and/or milk products, following the treatment methods. This aspect of the invention not only provides treatment methods for effectively alleviating and controlling diarrhea in affected animals, it is also beneficial for the health and safety of consumers of any food or nutrition-related products, e.g., meat and/or milk products, that are ultimately produced or derived from animals that have undergone the combination treatment for diarrhea according to the invention.

C. lechleri Proanthocyanidin Polymer and Rifaximin Combination Therapy and Applications of Use

The invention is directed to combination therapy for treating diarrhea, including diarrhea associated with pathogenic infection of neonatal, young and adult animals, which causes diarrhea. The methods also embrace the treatment of diarrhea induced by bacterial infection in combination with non-pathogenic causes in neonatal and young non-human animals, and in adult non-human animals, in need thereof, comprising administering to an animal in need of such treatment, a combination therapy comprising a pharmaceutically acceptable composition comprising a proanthocyanidin polymer from a Croton species or Calophyllum species in combination with rifaximin in amounts effective to treat the diarrhea. In preferred embodiments, the proanthocyanidin polymer is from a Croton species, namely, Croton lechleri. Treating the diarrhea in a non-human animal in need can involve reducing the severity and duration of the diarrhea in the animal. Treating the diarrhea can also involve increasing the survivability of the animal undergoing treatment. In an embodiment, the diarrhea is secretory or watery diarrhea. Treating or preventing diarrhea with the proanthocyanidin polymer from Croton lechleri in combination with rifaximin results in treating the diarrhea resulting from microbial infection, as well as in improving overall health of the animals so that their natural immune function can also assist in eradicating the infection, thereby reducing morbidity and mortality and increasing overall health and survival. The invention further provides methods of treating or preventing diarrhea by administration of rifaximin in the absence of the Croton spp. or Calophyllum spp. proanthocyanidin polymer compositions of the invention.

The combination therapy methods of the invention further include methods of preventing or reducing the incidence or severity of diarrhea in neonatal, young and adult animals having been exposed to or suspected of having been exposed to infection and infectious agents, which may cause or lead to diarrhea in the animals. For example, when one or a few animals in a population is diagnosed with an infection, the animals in the herd or other groups of animals that could have come into contact with the infected animal(s), or the feces or bodily fluids from such animal(s), may be administered a C. lechleri proanthocyanidin polymer composition and rifaximin combination therapy of the invention to prevent diarrheal disease or at least reduce the incidence or severity of the disease and/or the symptoms thereof. Alternatively, the methods of treatment may involve the administration of rifaximin in the absence of the C. lechleri proanthocyanidin polymer composition or extract.

The combination of a proanthocyanidin polymer formulation, composition, or botanical extract from Croton lechleri and rifaximin, or rifaximin alone, advantageously treats or prevents diarrhea in animals in need, particularly because of a more localized action of the combination in the enteric environment of the GI tract where potential bacterial causes of diarrhea are concentrated. In an embodiment, diarrhea treatment and prevention methods involve the use of a composition or formulation containing a proanthocyanidin polymer formulation, composition, or botanical extract from Croton lechleri and rifaximin. In an embodiment, a proanthocyanidin polymer formulation, composition, or botanical extract from Croton lechleri is administered to an animal in need thereof in combination with the administration of rifaximin to the animal. In an embodiment, both the proanthocyanidin polymer formulation, composition, or botanical extract from Croton lechleri and rifaximin comprise the formulation or composition that is administered to the animal in need of treatment. In another embodiment, the proanthocyanidin polymer formulation, composition, or botanical extract from Croton lechleri may be administered to the animal in need separately from the administration of rifaximin.

In embodiments in which a combination of the proanthocyanidin polymer formulation, composition, or botanical extract from Croton lechleri and rifaximin is administered to the animal in need thereof as separate formulations or compositions, the C. lechleri proanthocyanidin polymer formulation, composition, or botanical extract may be administered at the same time as the administration of rifaximin, or at a predetermined time before or after the administration of rifaximin to the animal, for example, immediately prior to or after the administration of rifaximin. In embodiments of the combination therapy of the invention, the C. lechleri proanthocyanidin polymer formulation, composition, or botanical extract is administered simultaneously with, before or after the administration of rifaximin. In other embodiments of the invention, rifaximin is administered before or after the administration of the C. lechleri proanthocyanidin polymer formulation, composition, or botanical extract.

In some embodiments, an interval of time, e.g., several minutes up to about an hour, may separate the administration of the C. lechleri proanthocyanidin polymer formulation, composition, or botanical extract from the administration of rifaximin to the animal. In an embodiment, a therapeutic dose of rifaximin is administered to the animal in a tablet dosage form in combination with a therapeutic dose of the C. lechleri proanthocyanidin polymer formulation, composition, or botanical extract, which may be administered in a tablet or non-tablet form, for example, a solution, gel, or paste composition or formulation.

The treatment regimen for a combination therapy according to the invention that is effective to treat diarrhea in a young or adult non-human animal may vary according to factors such as the disease state, age, and weight of the animal, and the ability of the therapy to elicit an anti-diarrhea response in the treated animal. It will be appreciated that while each of the embodiments of the invention may not be effective in achieving a positive therapeutic effect in every animal, a positive effect should be seen in a statistically significant number of animals as determined by a suitable statistical test known and accepted in the pertinent art, for example, without limitation, the Student's t-test or the chi2-test.

The terms “treatment regimen”, “dosing protocol” and dosing regimen are used interchangeably to refer to the dose and timing of administration of each therapeutic agent in a combination of the invention

In accordance with the invention, a C. lechleri-derived proanthocyanidin polymer, proanthocyanidin polymer composition, or botanical extract is considered a therapeutic agent and rifaximin is considered a therapeutic agent in the combination therapy. In an embodiment, each therapeutic agent of the invention may be administered either alone or in a pharmaceutical composition or formulation (also referred to as a medicament) that comprises the therapeutic agents and one or more pharmaceutically acceptable carriers, diluents, vehicles, or excipients, as generally used in standard pharmaceutical practice.

Each therapeutic agent in the combination therapy of the invention may be administered simultaneously, i.e., in the same pharmaceutical composition, concurrently, i.e., in separate pharmaceutical compositions administered one following the other in any order, or sequentially in any order. Sequential administration is particularly advantageous when the therapeutic agents in the combination therapy are in different dosing or dosage forms, e.g., when one therapeutic agent is in tablet or capsule form, and another agent is in the form of a gel, paste, or sterile liquid. In addition, sequential administration is also useful when the therapeutic agents in the combination are administered on different dosing schedules, e.g., if one agent is administered at least daily and the other agent is administered less frequently, such as once weekly, once every two weeks, or once every three weeks.

In some embodiments of the treatment method, the C. lechleri-derived proanthocyanidin polymer, proanthocyanidin polymer composition, or botanical extract (e.g., SB 300 or SP 303) is administered before the administration of rifaximin; in other embodiments of the treatment method, rifaximin is administered before the administration of the C. lechleri-derived proanthocyanidin polymer, proanthocyanidin polymer composition, or botanical extract.

In other embodiments, at least one of the therapeutic agents in the combination therapy of the invention is administered using the same dosage regimen (dose, frequency and duration of treatment) that is employed when the therapeutic agent is used as monotherapy for treating the diarrhea in young or adult animals. In other embodiments, the animal receives a lower total amount of at least one of the therapeutic agents in the combination therapy, compared with when the therapeutic agent is used as monotherapy, such as doses that are smaller, less frequent, and/or shorter in treatment duration.

In embodiments, each of the therapeutic agents in the combination therapy of the invention, or the rifaximin alone, can be administered orally or parenterally, including, without limitation, intravenous, subcutaneous, intramuscular, intraperitoneal, topical, rectal, intrauterine, injection, topical, and transdermal routes of administration.

In an aspect, a combination therapy of the invention, or rifaximin alone, is administered to a young or adult animal that has not been previously treated with an antidiarrheal agent, i.e., the animal is treatment-naïve. In another aspect, the combination therapy or rifaximin alone is administered to a young or adult animal that did not respond to a prior therapy or treatment, i.e., the animal is treatment-experienced.

In an embodiment of the invention, the methods are directed to treating diarrhea in non-human animals, including adult animals, neonates and young animals, e.g., animals infected with bacterial microorganisms (or exposed to or suspected of having been exposed to such microorganisms). In embodiments, the methods are directed to treating bacteria-infected newborns and young of livestock, domestic and farm animals, including grazing animals, which are oftentimes relatively large in size. In one embodiment, the immature animals to which treatment with the combination therapy of the invention or rifaximin alone is administered are neonatal (newborn) or infant animals, for example, one to ten hours after birth, one to fifteen hours after birth, twelve to twenty-four hours after birth, twenty-four to thirty-six hours after birth, one to three days after birth, one to four days after birth, one to six days after birth, or one to seven days after birth or up to two weeks after birth. Neonatal animals are generally those under two weeks of age. In an embodiment, the animals are treated with the therapeutic between day one and day four after birth. In some embodiments, the neonatal or young animals are treated one to five days of age, less than one week of age, or only a few weeks of age. In an embodiment, treatment occurs during the first weeks of life, for example, one to six weeks of age. In an embodiment, the animals are from two to ten weeks of age, for example, less than one, two, three, four, five, six, seven, eight, nine, or ten weeks of age. The animals undergoing treatment with the therapeutic of the invention may also be from one to four weeks of age, from one to six weeks of age, or from two to four weeks of age. In some embodiments, the animals are one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days old. In other embodiments, the animals are thirty to forty days old. In other embodiments, the animals are young animals, generally up to one year in age. In many cases, the animals are not weaned (unweaned), i.e., they are still drinking milk. For example, dairy calves are generally weaned at 60-80 days while beef cattle may be weaned at 3-8 months of age, pigs at 3 weeks of age, dogs at 7-8 weeks, and horses at 4 to 6 months of age. Also in many cases, neonatal is synonymous with unweaned. In some cases, the animals are newly weaned or weaned, but still juvenile, young, and non-adult. Such young animals are also highly susceptible to becoming afflicted with diarrhea from infectious agents such as bacteria and/or environmental causes.

According to the methods of the invention, neonatal and young animals, such as bacteria-infected animals, in need of treatment can be treated with a proanthocyanidin polymer from C. lechleri, e.g., SB 300, or a botanical extract derived from C. lechleri in combination with rifaximin, or with rifaximin alone, for one, two, three, four, five, six, seven, eight, nine, or ten days, etc. The C. lechleri proanthocyanidin polymer and rifaximin combination, or rifaximin alone, can be administered to the animal on consecutive days or intermittently, such as every other day, every two days, every three days, every four days, and the like. In an embodiment, the C. lechleri proanthocyanidin polymer and rifaximin combination, or rifaximin alone, is administered to the animals for three consecutive days. In an embodiment, the C. lechleri proanthocyanidin polymer and rifaximin combination, or rifaximin alone, is administered to neonatal animals between one and four days after birth for three consecutive days. As understood by the skilled practitioner, environmental, e.g., farm, conditions surrounding the neonatal and young animals may dictate the start and course of a treatment regimen such that the administration of the C. lechleri proanthocyanidin polymer and rifaximin combination, or rifaximin alone, occurs earlier in the animal's life and for a longer duration, especially since diarrheal disease typically affects neonatal and young animals in about the first seven days of life, or between about day one or day four of life. In the foregoing embodiments, animals particularly treatable with the combination of the invention are bovine or camel calves.

The methods of the invention further relate to the treatment of bacteria infected (or those exposed to or suspected of having been exposed to bacteria), adult, non-human animals, such as, without limitation, adult livestock, farm animals, and domestically or commercially used animals. As noted supra, often, but not always, the animals are large in size and have complex gastrointestinal systems. For example, in horses, which are optimally suited for the anti-diarrhea combination treatment methods of the invention, the intestinal volume of the animal is large, with the main site of diarrhea being in the colon. Consequently, the described methods provide treatment of such bacteria-infected animals, providing for an adequate amount and appropriate distribution of the C. lechleri proanthocyanidin polymer and rifaximin combination or rifaximin alone in the gut of the animal so as to treat the diarrhea and/or its symptoms and optimally cure the diarrhea in the animal.

In a related manner, ruminant animals, such as camels, which are also suited for the anti-diarrhea combination treatment methods of the invention, possess multi-chambered stomachs, e.g. four stomach compartments, including a rumen or first compartment of the alimentary canal, which serves as the primary site for microbial fermentation of ingested feed. The described methods provide treatment of ruminant animals such as camels, cows, sheep and goats with a combination of rifaximin and a proanthocyanidin polymer from C. lechleri, or composition thereof, or rifaximin alone, providing for an adequate amount and appropriate distribution of rifaximin and proanthocyanidin polymer in the GI tract/gut of the animal, rather than the rumen, so as to treat the diarrhea and/or its symptoms and optimally cure the diarrhea.

In an embodiment of the combination therapy methods of the invention, the combination of rifaximin and a proanthocyanidin polymer from C. lechleri, or rifaximin alone, is orally administered to adult animals that are infected with bacteria, in need of treatment for diarrhea, or that have been exposed or suspected to have been exposed to bacterial infection, having a large intestinal volume and/or multi-compartment stomachs and a rumen in a large volume and/or high concentration to treat the diarrhea. For example, the C. lechleri proanthocyanidin polymer, or composition thereof, and/or rifaximin is orally provided or administered to the animal in a volume of about 10-50 liters, or in a volume of 20-40 liters or in a volume of about 25-30 liters, so as to target and reach the animal's large intestine/gut for optimal treatment. In an embodiment in which ruminant animals are treated for diarrhea in accordance with the methods of the invention, rifaximin is administered in combination with the C. lechleri proanthocyanidin polymer, or composition thereof, formulated for oral administration to the animal such that the C. lechleri proanthocyanidin polymer bypasses the rumen and reaches the intestine for delivery of the composition to the affected area and to treat the diarrhea more efficiently. In adult ruminants there is a need to bypass the rumen to avoid dilution of the product in a large volume of liquid. The ruminants have specific anatomical structure, called the esophageal groove, in the stomach that closes and forms a tube which enables liquid to bypass the rumen. The closing of this esophageal groove is controlled by neural stimulation from suckling. Thus, bypassing the rumen may be achieved, for example, by using a bottle to administer the product.

In a particular embodiment, a formulation or composition comprising a botanical extract derived from C. lechleri, SB 300, or SP 303, is provided in combination with rifaximin in the form of a gel or paste formulation that is orally administered to the young or adult animal, such as, for example, a horse foal or adult horse, twice daily for three days, preferably, three consecutive days. In a particular embodiment, the twice daily doses are administered to the animal twelve hours apart. The paste formulation is particularly suitable as a product that acts locally in the gut and is minimally absorbed systemically. The paste product specifically addresses the normalization of stool formation and ion and water flow in the intestinal lumen of neonatal, young and adult animals, such as horses, and does not alter gastrointestinal motility, i.e., is not constipating. As but one mode of oral delivery, the paste formulation can be placed in the roof of the animal's mouth. In a particular embodiment, the paste formulation comprises beads (nano or microparticles) comprising enterically coated SB 300 or SP 303 and is orally administered to the animal. In an embodiment, the paste comprising SB 300 enteric beads is orally administered to an animal twice daily for three days. In some embodiments, the paste is orally administered for three consecutive days. In an embodiment, the paste comprising SB 300 enteric beads is orally administered to an animal in need at a dose of 2 mg/kg twice daily for three days. Such a formulation can also include rifaximin, for example a dosage of 1 mg/kg to 5 mg/kg or up to, and including, 10 mg/kg of rifaximin. The formulation is especially suitable for the normalization of stool formation in a short time period, e.g., less than a week or less than two weeks; for mitigation of weight loss; and reduction in supportive care costs, rehydration therapies, such as oral rehydration, in a treated animal afflicted with diarrhea. In some embodiments, a gel or paste formulation containing the C. lechleri proanthocyanidin polymer or botanical extract is administered to the animal, and/or rifaximin is also orally administered to the animal in a suitable dosage form, which may be, for example, a tablet form, such that both actives function to treat diarrhea in combination. In embodiments, the therapy may be administered with or without food.

The types of non-human neonatal, young and adult animals for which the treatment methods are suitable are not particularly limited as to animal type, genus, or species. In general, neonatal, young, or adult farm animals, food-source animals, livestock animals, animals bred or kept for various purposes, such as sport (e.g., racing, riding), transport, domestic, companion, industrial uses (e.g. hauling, pulling, plowing), and the like, are particularly amenable to treatment according to the methods of the invention. For example, encompassed by the methods of the invention is the treatment of bacteria-infected adult, neonatal or young non-human animals, such as cows (calves), cattle or steer (calves), camels (calves), sheep (lambs), rams, horses (foals), pigs (piglets), goats (kids), bison/buffalo (calves), llamas, donkeys, mules, yaks, etc. Neonatal, young and adult exotic animals, such as zoo animals of various species, are also embraced by the treatments of the invention. In an embodiment, the animals are grazing animals. The treatment of diarrhea in neonatal, unweaned and adult animals, for example, cows, steer, calves (bovine, camel, buffalo/bison), sheep, lambs, pigs, piglets, goats and foals (equine) with the combination of rifaximin and a C. lechleri proanthocyanidin polymer, polymer composition, or botanical extract is particularly embraced by the described methods.

In accordance with the described methods, the C. lechleri proanthocyanidin polymer composition reduces chloride flux across intestinal epithelial cells and reduces fluid movement into the intestinal lumen, which results in fluid loss and dehydration associated with secretory diarrhea. Therefore, the pharmaceutically acceptable formulations and methods of the invention are useful in prophylactic and therapeutic applications in the treatment of secretory diarrhea caused by infection, such as bacterial infection, or diarrhea caused by other microorganisms or factors, especially in preventing the symptoms of dehydration and electrolyte loss that accompany diarrhea, e.g., secretory/watery diarrhea.

In an embodiment, young animals treated by the methods of the invention are two to four weeks of age. In an embodiment, the animals are two to four week old calves, e.g., without limitation, bovine or camel calves, having diarrhea caused by infection with Salmonella, or crytosporidia or a combination thereof. In an embodiment, the animals are two to four week old calves, e.g., without limitation, bovine or camel calves, having undifferentiated diarrhea of unknown origin. In other embodiments, the animals treated by the methods of the invention are approximately 3-1000 kg in weight; or approximately 5-900 kg in weight, or approximately 10-350 kg in weight; or approximately 15-150 kg in weight; or approximately 25-60 kg in weight, or approximately 30-50 kg in weight, or approximately 30-40 kg in weight. In an particular embodiment, the young animal being treated for diarrhea is a bovine calf of approximately 20-40 kg in weight. In an particular embodiment, the young animal being treated for diarrhea is a camel calf of approximately 30-50 kg in weight.

In another embodiment, the adult animals treated by the methods of the invention are greater than four months of age. In a related embodiment, the animals have fully developed and competent GI tracts and colon function. In an embodiment, the animal has a weight (mass) of about 400-800 kg. In an embodiment, the animal has a weight of about 500-700 kg. In an embodiment, the animal has a weight of about 550-650 kg. In an embodiment, the animal has a weight of about 600 kg. In an embodiment, the animals have undifferentiated diarrhea of stress-induced origin.

Rifaximin alone or in combination with a proanthocyanidin polymer composition of the invention is administered in a therapeutically effective dose, for example, 10 mg-1000 mg, 100 mg-1000 mg, preferably from 20 mg to 750 mg, or 200 mg or 550 mg, or 200 mg to 600 mg. The dosage may depend upon the weight of the animal. The dosage of rifaximin may be anywhere from 0.1 mg/kg-100 mg/kg, as well as doses therebetween, including 1 mg/kg, 2 mg/kg, 3 mg/kg, 4 mg/kg, 5 mg/kg, 10 mg/kg or even 20 mg/kg.

In an embodiment, neonatal and young animals are treated prophylactically with the combination therapy of the invention comprising a C. lechleri proanthocyanidin polymer composition, such as SB 300 or SP 303, in enteric or non-enteric form, and rifaximin, or rifaximin alone, to prevent or reduce the risk or severity of the debilitating effects of diarrheal disease, such as bacteria-induced diarrhea, and its associated symptoms, e.g., dehydration and weight loss, in neonatal and young animals. According to the treatment method, the combination therapy comprising a C. lechleri proanthocyanidin polymer composition and rifaximin, or rifaximin alone, is administered to neonatal and young animals at a suitable time after birth to protect the animals from diarrhea outbreaks typically caused by infections and adverse environmental conditions. Administering the therapy to neonatal and young animals can also serve to ameliorate or reduce the risk of the animals' suffering from a more serious or severe form of diarrhea relative to animals that are not provided with therapy prior to or during an outbreak of disease or infection.

According to the invention, the C. lechleri proanthocyanidin polymer composition component of the combination therapy may be provided as an enteric or a non-enteric formulation and can be, for example, SB 300 or SP 303. The dose and regimen of C. lechleri proanthocyanidin polymer composition and the dose and regimen of rifaximin are within the skill of the practitioner to determine and will depend on the environmental conditions and health of the neonatal and young animals to be treated. The animals can be prophylactically treated with the therapy according to the invention, for example and without limitation, one to seven days, one to six days, one to four days, one to three days, or one or two days after birth. The treatment regimen can involve one, two, three, four, five, six, seven or more days, of therapy administration to the animals, modified or adjusted as necessary or desired, once or multiple times, e.g., twice, three or four times, per day. The animals can be regularly observed and monitored for health improvements and weight gain.

In an embodiment, the combination therapy of the invention comprising rifaximin and a C. lechleri proanthocyanidin polymer, composition, or botanical extract, or rifaximin alone, in amounts effective to control, reduce, and/or treat the diarrhea in non-human animals provides localized, non-systemic antidiarrheal activity in the gut of a treated animal. Such local, non-systemic activity of the rifaximin and C. lechleri proanthocyanidin polymer, composition, or botanical extract components of the treatment methods ultimately results in less than expected and/or unappreciable residual quantities of the components of the treatment methods, or component metabolic byproducts, found in animal-derived materials or food products, such as meat and/or milk. This aspect of the invention not only provides effective treatment methods for alleviating and controlling diarrhea in affected animals, it is also beneficial for the downstream health and safety of consumers of any food or nutrition-related products that are ultimately produced or derived from animals that have undergone treatment. In another embodiment, the lack of systemic absorption and the lack of dissemination from the enteric site of action of the agents, i.e., rifaximin and a C. lechleri proanthocyanidin polymer, composition, or botanical extract, and metabolic breakdown products thereof, may be associated with a corresponding lack, or at least less risk, of antibiotic resistance in a non-human animal treated according to the methods of the invention, as well as a lack, or less risk of potential downstream resistance or effects in consumers of any food or nutritional product derived from such treated animals.

### Physiologically and Pharmaceutically Acceptable Formulations

The C. lechleri proanthocyanidin polymer composition, in combination with rifaximin, or the rifaximin alone, can be provided in any physiologically, pharmaceutically, or therapeutically acceptable form. In an embodiment, each therapeutic agent may be separately formulated and administered in combination. The pharmaceutically acceptable composition, e.g., C. lechleri proanthocyanidin polymer and/or rifaximin, can be formulated for oral administration as, illustratively, but without limitation, powders; crystals; granules; small particles, including particles sized on the order of micrometers, e.g., microspheres and microcapsules; particles sized on the order of millimeters, particles sized on the order of nanometers, e.g., nanoparticles; beads; microbeads; pellets; pills; tablets; microtablets; compressed tablets or tablet triturates; molded tablets or tablet triturates; and in capsules, which are either hard or soft and contain the composition as a powder, particle, bead, solution or suspension. For combination therapy, the pharmaceutically acceptable composition comprising C. lechleri proanthocyanidin polymer and/or rifaximin can also be formulated for oral administration as a solution or suspension in an aqueous liquid, as a liquid incorporated into a gel capsule, as a gel, as a paste or gel paste, or as any other convenient formulation for administration. The combination therapy composition can be formulated for rectal administration, as a suppository, enema or other convenient form. The proanthocyanidin polymeric composition can also be provided as a controlled release system (See, e.g., Langer, 1990, Science 249: 1527-1533). The composition can be formulated as a dietary supplement or food supplement, e.g., as described in WO 00/47062, for administration to an animal in need thereof according to the present invention.

For the compositions of the invention, a pharmaceutically acceptable formulation can also include any type of pharmaceutically acceptable excipients, additives, carriers, or vehicles. By way of nonlimiting example, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose can be added to the proanthocyanidin polymer composition to increase the bulk of the composition. In addition, binders, such as, but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, can be added to the formulation to increase its cohesive qualities. Further, lubricants, such as, but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate and magnesium lauryl sulfate can be added to the formulation. Also, glidants, such as, but not limited to, colloidal silicon dioxide or talc can be added to improve the flow characteristics of a powdered formulation. Disintegrants, such as, but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch can also be added to facilitate disintegration of the formulation in the intestine.

In some embodiments of the combination therapy, the pharmaceutically acceptable formulations contain the C. lechleri proanthocyanidin polymer composition with an enteric coating, in addition to another pharmaceutically acceptable vehicle. In an embodiment, the proanthocyanidin polymer composition can be directly-compressed into a tablet. The tablet can be without excipients and of pharmaceutically acceptable hardness and friability, optionally, with a lubricant, e.g., without limitation, magnesium stearate, and enteric coated. In addition, rifaximin may be in tablet form, either in combination with the C. lechleri proanthocyanidin polymer in the same tablet, or in a separate tablet that is co-administered with the proanthocyanidin polymer. In another embodiment, the pharmaceutically acceptable compositions containing the proanthocyanidin polymer composition in combination with rifaximin alternatively include one or more substances that either neutralize stomach acid and/or enzymes or are active to prevent secretion of stomach acid. These formulations can be prepared by methods known in the art (See, e.g., methods described in Remington's “The Science and Practice of Pharmacy,” 22nd Edition, Editor-in-Chief: Lloyd V. Allen, Jr., Pharmaceutically acceptable Press, Royal Pharmaceutically acceptable Society, London, UK, 2013; and U.S. Pat. No. 7,323,195).

In an embodiment for use in combination therapy, the proanthocyanidin polymer composition is formulated with a substance that protects the proanthocyanidin polymer and/or the polymer composition from the stomach environment. For such protection, the proanthocyanidin polymer composition can be enteric coated. Enteric coatings are those coatings that remain intact in the stomach, but will dissolve and release the contents of the dosage form once it reaches the small intestine. A large number of enteric coatings are prepared with ingredients that have acidic groups such that, at the very low pH present in the stomach, i.e. pH 1.5 to 2.5, the acidic groups are not ionized and the coating remains in an undissociated, insoluble form. At higher pH levels, such as in the environment of the intestine, the enteric coating is converted to an ionized form, which can be dissolved to release the proanthocyanidin polymer composition. Other enteric coatings remain intact until they are degraded by enzymes in the small intestine, and others break apart after a defined exposure to moisture, such that the coatings remain intact until after passage into the small intestines. A variety of polymers are useful for the preparation of enteric coatings, and the application of an enteric coating to the proanthocyanidin polymer composition can be accomplished by any method known in the art for applying enteric coatings, as may be found, for example, and without limitation, in U.S. Pat. Nos. 7,323,195 and 7,341,744, incorporated herein by reference.

In another embodiment for use in combination therapy, the pharmaceutically acceptable composition of the proanthocyanidin polymer composition is formulated as enteric coated granules or powder (microspheres with a diameter of 300-500 microns) provided in either hard shell gelatin capsules or suspended in an oral solution for pediatric administration. The enteric coated proanthocyanidin polymer composition powder or granules can also be mixed with food, particularly for administration to neonatal or young animals. Such preparations may be prepared using techniques well known in the art. In addition, the proanthocyanidin polymer composition granules and powder can be prepared using any method known in the art, such as, but not limited to, crystallization, spray-drying or any method of comminution, preferably using a high speed mixer/granulator, as described, for example and without limitation, in U.S. Pat. No. 7,323,195, incorporated herein by reference.

In other embodiments for use in combination therapy, the proanthocyanidin polymer composition and/or rifaximin is in the form of an aqueous suspension in admixture with suitable excipients. Non-limiting examples of excipients that are suitable for the manufacture of aqueous suspension include suspending agents, for example, methylcellulose, sodium carboxymethylcellulose, hydroxy-propylmethylcellulose, sodium alginate, polyvinylpyrrolidone, gum tragacanth and gum acacia; dispersing or wetting agents, which may be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, e.g., sucrose, saccharin or aspartame.

Dispersible powders and granules suitable for the preparation of an aqueous suspension by the addition of water provide the proanthocyanidin polymer composition and/or rifaximin in admixture with a dispersing or wetting agent, suspending agent and one or more preservatives. Suitable dispersing or wetting agents and suspending agents are exemplified by those stated above. Additional excipients, for example, sweetening, flavoring and coloring agents, may also be present.

In yet another embodiment for use in combination therapy, the proanthocyanidin polymer composition and/or rifaximin is a gel or gel formulation. In an embodiment, the proanthocyanidin polymer composition is a paste formulation. In an embodiment, the paste formulation contains a purified botanical extract derived from C. lechleri. In another embodiment, the paste formulation contains enterically coated beads comprising SB 300 or SP 303. In an embodiment, the paste formulation contains enteric protected SB 300 beads. In an embodiment, the gel or paste is contained or preloaded in a delivery device, such as a syringe, e.g., a needle-less syringe, or other type of applicator or delivery system, especially for oral delivery. A gel or paste formulation is particularly suited for administration to bacteria-infected neonatal and young foals, but also is applicable for other bacteria-infected adult and neonatal animals, such as those described herein. In an embodiment, the gel or paste is not contained in a delivery device for combination therapy, but is administered to the roof of the mouth of the animal, particularly one that is too incapacitated or ill to eat or drink, thereby eschewing an oral or other mode of administration. In an embodiment, the gel comprises pH-sensitive polymeric particles, such as microparticles or nanoparticles, to allow for pH-dependent uptake of the active compound into cells and/or the pH-dependent release of the active compound in different pH environments in an animal. Processes for generating granules and particles comprising the proanthocyanidin polymer composition or a compressible form thereof for use in combination therapy are as known and practiced in the art, and as provided, for example, in U.S. Pat. No. 7,341,744, the contents of which are incorporated by reference herein. In an embodiment, gels are prepared for oral delivery and contain copolymers, such as poloxamers and Pluronics of different types, e.g., Pluronic F.

In another embodiment for use in the therapy of the invention, the proanthocyanidin polymer composition and/or rifaximin is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent. In an embodiment, the paste formulation contains beads with enterically coated SB 300 or SP 303, which is administered to an animal, such as, for example, a foal or adult horse, at a dose of 2 mg/kg. More particularly, the paste formulation containing enterically coated SB 300 beads is administered to the animal at a dose of 2 mg/kg, twice a day for three days. In an embodiment, the paste containing enteric protected SB 300 beads is administered twice a day at twelve hour intervals.

Oily suspensions may be formulated by suspending the C. lechleri proanthocyanidin polymer as active ingredient in a vegetable oil, e.g., arachis oil, olive oil, sesame oil or coconut oil, or in mineral oil, such as liquid paraffin. The oily suspensions may contain a thickening agent, e.g., beeswax, hard paraffin or cetyl alcohol. Oral preparations can include sweetening agents as mentioned above and flavoring agents to improve palatability. Pharmaceutically acceptable preservatives, for example, an anti-oxidant such as ascorbic acid, can also be added to such compositions.

The C. lechleri proanthocyanidin polymer pharmaceutical compositions used in the combination therapy methods of the invention may also be in the form of an oil-in-water emulsions. The oily phase may be a vegetable oil such as olive oil or arachis oil, or a mineral oil such as liquid paraffin or mixtures of these oils. Examples of emulsifying agents include, without limitation, naturally-occurring phosphatides, e.g., soy bean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Sweetening, coloring and flavoring agents can be included in the emulsions.

Syrups and elixirs containing the C. lechleri proanthocyanidin polymer and/or rifaximin for use in the therapy of the invention may also can be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, orally deliverable or administrable aqueous or oleagenous suspension. This suspension may be formulated according to methods known in the art using suitable dispersing or wetting agents and suspending agents, such as those mentioned above. The sterile pharmaceutical preparation may also be a sterile solution or suspension in a non-toxic parenterally-acceptable diluent or solvent, for example, a solution in 1,3-butane diol. Illustrative, acceptable vehicles and solvents that may be used in the preparations include water, Ringer's solution and isotonic sodium chloride solution. Co-solvents, e.g., ethanol, propylene glycol or polyethylene glycols, may also be included. In addition, sterile, fixed oils, e.g., any bland, fixed oil such as synthetic mono- or diglycerides, are conventionally employed as solvents or suspending media and may be used. In addition, fatty acids, such as oleic acid and the like, may be used in injectable preparations.

### Dosage Forms and Administration

In a particular embodiment for treating diarrhea in neonatal animals, e.g., without limitation, bovine and camel calves, foals, kids, lambs, etc., the C. lechleri proanthocyanidin polymer composition for use in combination therapy is in powder, e.g., reconstitutable powder, form. The proanthocyanidin polymer composition may be enterically coated or not enterically coated. In an embodiment, the neonates are less than one week in age. In an embodiment, the neonatal animals are bovine calves or camel calves.

In a particular embodiment for treating diarrhea in adult animals, e.g., without limitation, horses, camels, llamas, cows, sheep, bison, etc., the proanthocyanidin polymer composition and/or rifaximin is in powder, e.g., reconstitutable powder, form for use in therapy. The composition may be enterically coated or not enterically coated. In an embodiment, the non-human adult animal is of an age that its large intestine/bowel and colon are fully competent and functional. In an embodiment, the non-human animal is greater than four months of age. In an embodiment, the non-human animals are adult horses or camels. In an embodiment, the animals needing therapy of the invention are afflicted with bacteria-induced diarrhea. In an embodiment, the animal further experiences involvement of one or more of another bacterial, e.g., Clostridium spp. infection, a viral infection, e.g., rotavirus and/or coronavirus, whose mechanism of action involves infection and subsequent destruction of the cells lining the intestinal tract, or a parasitic infection, e.g., nematodes.

In an embodiment, the powder form of the proanthocyanidin polymer composition and/or rifaximin is reconstituted or mixed with liquid, such as oral electrolytes, milk or a milk replacer, water, physiological saline, to produce a liquid form or suspension. Milk replacer is generally a source of protein from different origins (for example, milk from a different species, soy, or eggs) and energy (lactose and fat) given to the calf or other animals to replace milk from the mother. In a specific embodiment, the proanthocyanidin polymer composition is mixed at 200-800 mg per kg of the powder milk replacer prior to reconstitution. In an embodiment, the powder form of the proanthocyanidin polymer composition is provided in the form of individual dosages in packets, e.g., packaged dosage forms, wherein some number of individual packets are provided for use in a treatment regimen. In certain embodiments, the packaged dosage form contain 50-600 mg of the proanthocyanidin polymer composition, preferably, 200-300 mg of the proanthocyanidin polymer composition. The number of individual doses that can be packaged and provided together is not intended to be limiting, and can include, for example, one to twenty packaged doses; one to ten packaged doses; two, four, six, eight, ten, or more packaged doses, as well as numbers of packaged doses in-between the foregoing, for efficiency of use, handling and for commercial efficacy. Those skilled in the art will appreciate that due to the higher purity of compositions such as SP-303 or crofelemer and SB-300, more by weight of SB-300 than SP-303 will need to be used in formulations to achieve the same amount of the active ingredient of the proanthocyanidin polymer composition. SB-300 generally has about 67% by weight of the proanthocyanidin polymer composition while SP-303 has higher purity, for example 99-100%. The dosage of rifaximin may be from 1-1000 mg, in part, depending upon the weight of the animal to be dosed, resulting in certain cases is dosages of 0.5 mg/kg to 10 mg/kg.

In another embodiment, the powder form of the proanthocyanidin polymer composition and/or rifaximin is provided in a container, such as a bag, box, bucket, or pail (e.g., 5 lb. to 25 lb. pails), in which the powder can be in an amount of, for example, 100 grams (g) or more, and can optionally include a measuring device, such as a scoop, cup, spoon, trowel, dipper, or ladle. Such containers encompass, for example, an individual daily dose of the composition; or an amount suitable for multiple treatments, e.g., a two-day treatment, three-day treatment, four day treatment, etc. An effective amount of the powder can also be mixed with feed for consumption by the young animals, e.g., calves, in need thereof. In certain embodiments the proanthocyanidin polymer composition and/or rifaximin is administered in an amount of 1-8 mg/kg twice a day for 3-5 days. In certain embodiments, the rifaximin is administered in an amount of 1-10 mg/kg two or three times per day for 3 to 5 days or more.

In another embodiment, the C. lechleri proanthocyanidin polymer composition and/or rifaximin is administered or delivered to a neonatal or adult animal afflicted with diarrhea and in need thereof by providing the compound as a bolus or pill. In an embodiment, the proanthocyanidin polymer composition formulated as bolus, i.e., a pill, capsule, or tablet, is orally administered to the animals afflicted with diarrhea or symptoms thereof directly in the mouth. In a particular embodiment, the treatment regimen comprises administering a dose of 250 mg of the product, e.g., as embraced by one bolus per sick animal for a determined time period, for example, for one, two, or three or more days. The product can be provided to an animal in need thereof in portions of the complete dose, in which the portions are administered one or two or more times per day. Alternatively, the complete dose can be administered to an animal in need thereof one or two or more times per day. In a particular embodiment, the treatment encompasses a dose of 250 mg given two times a day. In another embodiment, the combination therapy encompasses an oral bolus dose of 250 mg of the C. lechleri proanthocyanidin polymer given two times a day for 3 days. In an embodiment, the dose is the C. lechleri proanthocyanidin polymer composition, SB 300, in enteric form or in non-enteric form, e.g., a reconstituted powder form, for use in the combination therapy of the invention.

The routes of administration are not intended to be limiting. Illustratively, administration of the combination therapy may be via any suitable, convenient or preferred route of administration including oral, buccal, dental, periodontal, via food source (animal feed), nutrition source, or libation source, otic, inhalation, endocervical, intramuscular, subcutaneous, intradermal, intracranial, intralymphatic, intraocular, intraperitoneal, intrapleural, intrathecal, intratracheal, intrauterine, intravascular, intravenous, intravesical, intranasal, ophthalmic, biliary perfusion, cardiac perfusion, spinal, sublingual, topical, transdermal, intravaginal, rectal, ureteral, or urethral. In certain embodiments, oral, buccal, and food and/or drink supplement are particularly suitable routes. In an embodiment, the product is an aqueous formulation and is provided to the animal as a drench or directly from a ready-to-use (RTU) bottle directed to the esophageal cavity so as to more effectively reach the animal's intestine/gut for optimal activity. In a related embodiment, administration can also be by inclusion in the regular or special diet of the animal, such as in a functional food for the animals or companion animals.

Dosage forms of the combination therapy can include, without limitation, oral, injectable, transdermal, aerosol including metered aerosol, chewable products or pellets, capsules, capsule containing coated particles, nanoparticles, or pellets, capsule containing delayed release particles, capsule containing extended release particles, concentrates, creams and augmented creams, suppository creams, discs, dressings, elixirs, emulsions, enemas, extended release films or fibers, gases, gels, metered gels, granules, delayed release granules, effervescent granules, implants, inhalants, injectable lipid complexes, injectable liposomes, inserts or devices, extended release inserts, intrauterine devices, jellys, liquids, extended release liquids, lotions, augmented lotions, oils, ointments, augmented ointments, pastes, pastilles, pellets, powders, reconstituted powders, extended release powders, metered powders, solutions, drops, concentrated solutions, gel forming solutions/drops, sponges, sprays, metered sprays, suppositories, suspensions, suspensions/drops, extended release suspensions, syrups, tablets/pills, chewable tablets/pills, tablets/pills containing coated particles, delayed release tablets/pills, dispersible tablets/pills, effervescent tablets/pills, extended release tablets/pills, orally disintegrating tablets/pills, tapes, or troches/lozenges. The dosages can be provided as formulations, compositions, pharmaceutically acceptable formulations and compositions, physiologically acceptable formulations and compositions, including pharmaceutically and physiologically acceptable carrier, excipients, diluents, or vehicles as known and used in the art.

For oral administration of the combination therapy, the C. lechleri proanthocyanidin polymer product, or a composition thereof, may be encapsulated and formulated with suitable carriers, and the like, and administered in solid dosage forms in combination with rifaximin. Nonlimiting examples of suitable carriers, excipients, diluents and vehicles include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, edible oils, and the like. The formulations can also include lubricating agents, wetting agents, emulsifying and suspending agents, preserving agents, sweetening agents or flavoring agents. The compositions can be formulated to provide rapid, sustained, extended, or delayed release of the active ingredient after administration to the animal by employing protocols and methods well known in the art. The formulations can also include compounds or substances that reduce proteolytic degradation and promote absorption such as, for example, surface active agents.

As will be appreciated by those having skill in the art, the specific doses can be calculated according to the approximate body weight, body mass, or body surface area of the animal, or the volume of body space or mass to be occupied. The dose also depends on the particular route of administration selected by the practitioner. Further refinement of the calculations necessary to determine an appropriate dosage for treatment is routinely made by those of ordinary skill in the art, for example, using appropriate assays and analytical procedures, such as has been described for certain compounds (e.g., Howitz et al., Nature, 425:191-196, 2003). Exact dosages can be determined based on standard dose-response studies. Therapeutically effective doses for treatment of afflicted animals can be determined, by titrating the amount of the active product given to the animal to arrive at the desired therapeutic effect, while minimizing side effects.

For use in treating diarrhea and its symptoms in neonatal and young animals in accordance with the combination therapy methods of the invention, a therapeutically acceptable form of the C. lechleri proanthocyanidin polymer composition and/or rifaximin is administered, particularly orally administered, in an amount ranging from 0.1 to 100 mg/kg per day, once, twice or more daily. In other embodiments, the amount can range from about 0.1 to about 10 mg/kg/day, once, twice or more daily; or from about 0.1 to about 25 mg/kg/day, once, twice or more daily; or from about 0.1 to about 30 mg/kg/day, once, twice or more daily; or from about 0.1 to about 40 mg/kg/day, once, twice or more daily. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between. In still other embodiments, the amount can range from about 1 to about 10 mg/kg/day once, twice or more daily; or from about 1 to about 5 mg/kg/day, from about 1 to about 8 mg/kg/day, from about 1 to about 10 mg/kg/day, or from about 2 to about 4 mg/kg/day once, twice or more daily. In other embodiments, the foregoing amounts of the C. lechleri proanthocyanidin polymer composition and/or rifaximin are administered, for example, twice daily, three times daily, four times daily, or more than four times daily, rather than once per day. Higher doses, e.g., 50 mg/kg or 100 mg/kg per day or twice or more daily, may be required, as necessary, to treat diarrhea and accompanying dehydration in the neonatal and young animals.

For use in treating diarrhea and its symptoms in adult animals in accordance with the methods of the invention, a therapeutically acceptable form of the C. lechleri proanthocyanidin polymer, including a C. lechleri botanical extract, is administered, particularly orally administered, in combination with rifaximin, or rifaximin alone is administered in an amount ranging from 0.1-100 mg/kg per day. In other embodiments, the amount can range from about 0.1 to about 10 mg/kg/day; or from about 0.1 to about 25 mg/kg/day, or from about 0.1 to about 30 mg/kg/day, or from about 0.1 to about 40 mg/kg/day. In embodiments of the invention, the amount of rifaximin in the combination therapy may be in an amount ranging from 0.1-100 mg/kg per day. In other embodiments, the amount of rifaximin can range from about 0.1 to about 10 mg/kg/day; or from about 0.1 to about 25 mg/kg/day, or from about 0.1 to about 30 mg/kg/day, or from about 0.1 to about 40 mg/kg/day. In specific embodiments, the amount of rifaximin is 50 mg, 100 mg, 150 mg, 200 mg, 250 mg, 300 mg, 350 mg, 400 mg, 500 mg, or greater, e.g., to 1000 mg, including milligram amounts in between.

In an embodiment for use in combination therapy, the dose of the C. lechleri proanthocyanidin polymer, including a C. lechleri botanical extract, is 2 mg/kg twice a day. In an embodiment, the 2 mg/kg dose is administered twice a day for three days. Higher doses, e.g., 50 mg/kg/day, 100 mg/kg/day, 200 mg/kg/day, or greater, may be required, as necessary, to treat the diarrhea and accompanying dehydration in adult animals. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between.

In other embodiments, for the treatment methods involving the combination therapy, a suitable dose for the C. lechleri proanthocyanidin polymer product, or the C. lechleri proanthocyanidin polymer composition, such as SP 303 or SB 300, and/or for rifaximin, may range from about 1 mg to about 1000 mg, either daily or multiple times per day. In an embodiment, a suitable dose may range from about 10 mg to about 500 mg, either daily or multiple times per day. In an embodiment, a suitable dose may range from about 50 mg to about 350 mg, either daily or multiple times per day. In an embodiment, a suitable dose may range from about 30 mg to about 400 mg, either daily or multiple times per day. In an embodiment, a suitable dose may range from about 100 mg to about 250 mg, either daily or multiple times per day. In an embodiment, a suitable dose may range from about 50 mg to about 300 mg, either daily or multiple times per day. The doses administered multiple times per day can be given for consecutive days, e.g., two days, three days, four days, five days, six, days, seven days, or more, in some embodiments. A dose administered multiple times per day may embrace two, three, four, five, six, or more times per day. It will be understood that the amount and dose ranges described herein include the lower and higher amounts specified, as well as amounts in between.

Other dosing schedules, such as every other day, or every third day, every fourth day, etc. are embraced by the invention. In addition, one having skill in the art will appreciate that doses and amounts administered to the animal can vary, given the wide range of weights of the animals undergoing treatment, as well as the animal species and type of digestive system, e.g., ruminant or non-ruminant. In an embodiment the C. lechleri proanthocyanidin polymer is SB 300. In an embodiment the C. lechleri proanthocyanidin polymer is enterically coated SB 300. In an embodiment the C. lechleri proanthocyanidin polymer is non-enterically coated SB 300.

In some embodiments of the combination therapy of the invention, daily doses, including multiple daily doses, e.g., twice or three times a day, of the C. lechleri proanthocyanidin polymer product, and/or rifaximin, may be 2 mg, 5 mg, 10 mg, 15 mg, 20 mg, 25 mg, 50 mg., 100 mg, 150 mg, 175 mg, 200 mg, 225 mg, 250 mg, 275 mg, 300 mg, 325 mg, 350 mg, 375 mg, 400 mg, 500 mg (or there between) per animal. Administration schedules may also be altered to achieve a therapeutically effective concentration of the C. lechleri proanthocyanidin polymer to treat the diarrhea and its symptoms as described herein. By way of specific, yet nonlimiting example, a suitable dosage amount of the therapeutic agent for use in the methods according to the invention is 200 mg or 250 mg administered once or twice daily. In some embodiments, the therapeutic compound may be administered once per day, twice per day, thrice per day, 4 times per day, 5 times per day, 7 times per day or 10 times per day. Often the therapeutic dosage is divided into equal parts administered throughout the day, however in some embodiments related to treating more severe or entrenched symptoms, it may be useful to tailor the dosage administration schedule so that most of the daily treatment is administered at a predetermined time of the day, e.g., the beginning half of the day. In some embodiments, about 50% 60%, 70% or 80% of the dosage is administered in the first half of the day. In other embodiments, it may be more appropriate to administer most of the dosage in the latter half of the day so that about 50%, 60%, 70% or 80% of the dosage is administered in the latter half of the day.

It will be understood that the dose amount actually administered can be determined by the practitioner, in the light of the relevant circumstances, including the severity of the disease, condition, or symptoms thereof being treated, the form of the product to be administered, the age, weight, and response of the individual animal receiving treatment, as well as the chosen route of administration.

The methods of the invention further embrace the administration of pharmaceutically acceptable formulations of the proanthocyanidin polymer composition or rifaximin either alone or in combination with other supplements or agents for treatment or amelioration of the symptoms of secretory diarrhea, such as rehydration agents, electrolytes (e.g., sodium, potassium, magnesium, chloride and formulations thereof), antibiotics, gut-lining protectants, such as kaolin, pectin, or bismuth liquid, and fluid adsorbents, such as attapulgite. Other agents may include anti-motility agents, although because many of the microorganisms and pathogens that are associated with diarrhea induction in neonatal and young animals concomitantly decrease gut motility, the use of anti-motility drugs may be contraindicated. Natural biological products, e.g., Lactobacillus or Streptococcus faecium, or probiotics, may also be employed as additives to restore the natural balance of intestinal flora in affected neonatal, young, or adult animals.

### EXAMPLE

### Example 1

A representative paste composition comprising enterically coated SB 300 beads for use in the combination therapy with rifaximin according to the present invention is presented in this Example. For administration to animals and as noted hereinabove, the paste containing enteric SB 300 beads may be contained in a syringe. A paste containing enteric coated SB 300 beads may contain the following components:

| Component | % w/w | Theoretical mg/syringe |
|---|---|---|
| SB 300 enteric beads | 21.91 | 3286.6* |
| Vegetable oil | 64.42 | 9663.5 |
| Cetyl alcohol | 9.76 | 1464.2 |
| Apple flavor | 0.08 | 11.7 |
| Silicon dioxide | 2.73 | 410.0 |
| Butylated hydroxytoluene | 0.04 | 5.9 |
| Titanium dioxide | 1.05 | 158.1 |
| Total | 100.0 | 15000 |

*3286.6 mg SB 300 enteric beads corresponds to 880 mg theoretical SB 300.

Rifaximin may be administered to the animal as a solid dosage form, e.g., 200 mg or 550 mg, in combination with the SB 300 product, or with a C. lechleri proanthocyanidin polymer composition, e.g., SP 303.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

What is claimed is:

1. A method of treating diarrhea in a non-human animal, the method comprising orally administering to the non-human animal in need thereof a combination therapy comprising an aqueous soluble proanthocyanidin polymer from Croton lechleri and the antimicrobial agent rifaximin in amounts effective to treat the diarrhea in the non-human animal.

2. The method according to claim 1, wherein the C. lechleri proanthocyanidin polymer is administered as an enteric coated pharmaceutical composition.

3. The method according to claim 1, wherein the C. lechleri proanthocyanidin polymer is administered as a non-enteric pharmaceutical composition.

4. The method according to any one of claims 1 to 3, wherein the non-human animal is a juvenile animal.

5. The method according to any one of claims 1 to 3, wherein the non-human animal is an adult animal.

6. The method according to any of claims 1 to 5, wherein the non-human animal is selected from cattle, horses, camels, bison, buffalo, sheep, pigs, goats, rabbits, dogs and cats.

7. The method according to any one of claims 1 to 6, wherein the C. lechleri proanthocyanidin polymer is administered in an amount of 250 mg twice daily in combination with rifaximin.

8. The method according to any one of claims 1 to 7, wherein rifaximin is administered in a solid dosage form in combination with the C. lechleri proanthocyanidin polymer.

9. The method according to any one of claims 1 to 8, wherein rifaximin is administered in a dose of 20 mg to 1000 mg.

10. The method according to any one of claims 1 to 9, wherein rifaximin is administered in a dose of 200 mg or 550 mg.

11. The method according to any one of claims 1 to 10, wherein the C. lechleri proanthocyanidin polymer is administered as powder reconstituted with a liquid selected from oral electrolytes, milk, milk replacer, physiological saline, or water in combination with rifaximin.

12. The method according to any one of claims 1 to 11, wherein the C. lechleri proanthocyanidin polymer is administered as a bolus in combination with rifaximin.

13. The method according to any one of claims 1 to 12, wherein the C. lechleri proanthocyanidin polymer is administered in animal feed or drink.

14. The method according to any one of claims 1 to 13, wherein the C. lechleri proanthocyanidin polymer is formulated in the form of a gel, paste, or gel paste.

15. The method according to claim 14, wherein the gel, paste, or gel paste is administered to the animal by topical application to the roof of the animal's mouth.

16. The method according to claim 14 or claim 15 wherein the gel, paste, or gel paste is contained in a delivery device.

17. The method according to claim 16, wherein the delivery device is a syringe.

18. The method according to any one of claims 14 to 17, wherein the gel, paste, or gel paste comprises polymeric microparticles or nanoparticles containing the C. lechleri proanthocyanidin polymer in combination with rifaximin.

19. The method according to claim 18, wherein the polymeric microparticles or nanoparticles are pH-sensitive.

20. The method according to any one of claims 1 to 19, wherein the C. lechleri proanthocyanidin polymer is administered to the animal in an amount of at least 50 mg to 250 mg and rifaximin is administered to the animal in an amount of at least 50 mg to 600 mg.

21. The method according to any one of claims 1 to 20, wherein symptoms associated with the diarrhea in the non-human animal include dehydration and electrolyte loss.

22. The method according to any one of claims 1 to 21, wherein the C. lechleri proanthocyanidin polymer composition is selected from the group consisting of SB 300, SP 303 and crofelemer.

23. The method according to claim 22, wherein the C. lechleri proanthocyanidin polymer composition is SB 300.

24. The method according to claim 18, wherein the paste comprises enteric coated SB 300 and is administered to the animal in an amount of 2 mg/kg twice a day for three days.

25. The method according to claim 24, wherein the paste is administered twice a day, twelve hours apart.

26. The method according to any one of claims 1 to 25, wherein the rifaximin is administered to the animal at the same time as the aqueous soluble proanthocyanidin polymer from Croton lechleri is administered.

27. The method according to any one of claims 1 to 25, wherein the rifaximin is administered to the animal before the aqueous soluble proanthocyanidin polymer from Croton lechleri is administered.

28. The method according to any one of claims 1 to 25, wherein the rifaximin is administered to the animal after the aqueous soluble proanthocyanidin polymer from Croton lechleri is administered.

29. The method according to any one of claims 1 to 25, wherein a composition comprising effective amounts of the aqueous soluble proanthocyanidin polymer from Croton lechleri and rifaximin and a pharmaceutically acceptable carrier, vehicle, or excipient is administered to the animal.

30. A kit comprising a first container, a second container and a package insert, wherein the first container comprises at least one dose of a medicament comprising an aqueous soluble proanthocyanidin polymer from Croton lechleri; the second container comprises at least one dose of a medicament comprising rifaximin; and the package insert comprises instructions for use in treating a non-human animal for diarrhea.

31. A method for treating bacteria-induced diarrhea in a young or adult non-human animal, said method comprising administering to the animal a combination therapy which comprises an aqueous soluble proanthocyanidin polymer from Croton lechleri and rifaximin in amounts effective to treat the diarrhea.

32. The method according to any one of claims 1 to 31, wherein the combination of rifaximin and a C. lechleri proanthocyanidin polymer, composition, or botanical extract thereof provides localized and targeted activity against diarrhea-causing pathogens in the enteric environment of the GI tract of the animal undergoing treatment and is not systemically absorbed.

33. The method according to claim 32, wherein consumable products derived or produced from a non-human animal treated with the combination of rifaximin and a C. lechleri proanthocyanidin polymer, composition, or botanical extract thereof contain insignificant residual amounts of the rifaximin and/or the C. lechleri proanthocyanidin polymer, composition, or botanical extract components, or breakdown products thereof, of the combination therapy.

34. The method according to claim 32, wherein resistance to the rifaximin and/or the C. lechleri proanthocyanidin polymer, composition, or botanical extract components, or breakdown products thereof, of the combination therapy is minimized or absent in the treated non-human animals and in consumable products derived therefrom.

35. A method of treating diarrhea in a non-human animal, the method comprising orally administering to the non-human animal in need thereof a composition comprising rifaximin in amounts effective to treat the diarrhea in the non-human animal.

36. The method according to any one of claim 35, wherein the non-human animal is a juvenile animal.

37. The method according to any one of claim 35, wherein the non-human animal is an adult animal.

38. The method according to any of claims 35 to 37, wherein the non-human animal is selected from cattle, horses, camels, bison, buffalo, sheep, pigs, goats, rabbits, dogs and cats.

39. The method according to any one of claims 35 to 38, wherein rifaximin is administered in a dose of 20 mg to 1000 mg.

40. The method according to any one of claims 35 to 39, wherein rifaximin is administered in a dose of 200 mg or 550 mg.

41. The method according to any one of claims 35 to 40, wherein rifaximin is administered in animal feed or drink.

42. The method according to any one of claims 35 to 41, wherein symptoms associated with the diarrhea in the non-human animal include dehydration and electrolyte loss.

43. A method for treating bacteria-induced diarrhea in a young or adult non-human animal, said method comprising administering to the animal rifaximin in an amount effective to treat the diarrhea.

44. The method according to any one of claims 35 to 43, wherein rifaximin provides localized and targeted activity against diarrhea-causing pathogens in the enteric environment of the GI tract of the animal undergoing treatment and is not systemically absorbed.

45. The method according to claim 44, wherein consumable products derived or produced from a non-human animal treated with rifaximin contain insignificant residual amounts of the rifaximin or breakdown products thereof.

46. The method according to claim 44, wherein resistance to the rifaximin is minimized or absent in the treated non-human animals and in consumable products derived therefrom.
